(19) United States
(12) Patent Application Publication
(10) Pub. No.: US 2021/0251610 A1
(43) Pub. Date: Aug. 19, 2021

(54) AUTOMATED DIAGNOSTICS IN 3D ULTRASOUND SYSTEM AND METHOD

(71) Applicant: Her Majesty The Queen in Right of Canada, as Represented by The Minister of National Defence, Ottawa (CA)

(72) Inventors: Stergios STERGIOPOULOS, North York (CA); Mahdi MARSOUSI, Maple (CA); Konstantinos PLATANIOTIS, Toronto (CA)

(21) Appl. No.: 16/708,964

(22) Filed: Dec. 10, 2019

Related U.S. Application Data
(60) Provisional application No. 62/777,343, filed on Dec. 10, 2018.

(51) Int. Cl. A61B 8/08; A61B 8/13; G06N 3/08; G16H 50/20

(52) U.S. Cl. CPC A61B 8/5223; A61B 8/483; G16H 50/20; G06N 3/08; A61B 8/13

(57) ABSTRACT

A 3D image is provided for processing. A current resolution is set to a first coarse resolution and the current data is set to a known current data. A process is then iterated and within each iteration of the process the following is performed: pre-processing the ultrasound image in accordance with the current resolution; providing the pre-processed ultrasound image and the current data to a trained system for extracting therefrom objects of interest at the current resolution, the objects of interest extracted with a likelihood above a known threshold; determining data relating to each extracted object of interest to result in first current data; when the current resolution is a finest resolution, stopping the iterative process; and when the current resolution is other than a finest resolution, setting the current resolution to a finer resolution and returning to the start of the iterative process.

### FIELD OF THE INVENTION

The invention relates to the field of imaging and more particularly to a novel architecture and method for analysing data from an ultrasound dataset.

### BACKGROUND

Approximately 90% of trauma deaths occur in an accident zone prior to medical or surgical interventions. This used to be the obvious result of untreatable massive injury, but advances in medicine now allow a lot of traumatic injuries to be treated when diagnosed and addressed early enough.

Unfortunately, the lack of intelligent diagnostic tools that are capable of providing rapid and accurate diagnosis of non-visible internal injuries is the major challenge facing medical personnel, especially in mass casualty situations, under-served regions, and far forward operations within the defense sector. To this day, there is no portable system that provides relevant image data and automated diagnostic tools for use at the site of a trauma. Such a system with no or minimal training requirements should be capable of detecting life threatening injuries within the so-called “golden hour” of trauma diagnosis. The stress, commotion and the non-specific signs—symptoms—of trauma and the variability of patient reactions to injury result in frequently unreliable physical examinations in trauma settings. This in turn has been known to lead to catastrophic results.

Ultrasounds have been widely used for medical diagnosis in hospital and doctor's offices. “Pre-hospital” ultrasound has been used in emergency ambulances and helicopters mainly in North America and Central Europe since the late 1990s. The use of “in field” ultrasound has also been considered for mass casualty incidents. In all cases, use of ultrasound equipment is restricted to specialists or well-trained staff, which are not available in most emergency crews. Although ultrasound systems with a high degree of mobility—smart-phone-sized, PDA based-systems, e.g. VSCAN™ or Signos™—are available, these systems have limited diagnostic utility, offer no automated diagnosis options, and cannot be used reliably by first responders. In practice, experienced first responders are using their long-term experience and knowledge during triage. More specifically, to find a relevant internal anatomical region using 2D ultrasound imaging, placement of an ultrasonic probe and the assessment of a 2D image—B-scan image—requires training and long-term experience. This is simply not possible in typical paramedic situations.

As well as for a civilian emergency care market, both the US and Canadian Armed Forces desire a field-deployable compact 4D (3 spatial dimensions+Time) ultrasound imaging system capable of providing rapid diagnosis and triage of non-visible internal injuries. The need for such a system has been identified by both the Canadian Forces Surgeon General and the US-Army Director of the Combat Casualty Care Program. NATO allies, U.K. and Germany have issued similar requirements. Unfortunately, a portable and field operable real time 3D ultrasound imaging system is unavailable.

The rapid diagnosis of invisible internal injury in an austere, rushed, or low-tech environment remains a challenge for medical personnel and search and rescue personnel. The availability of a portable real-time 3D ultrasound imaging system with semi-automated or fully automated diagnostic capabilities for detecting non-visible internal abdominal bleeding, pneumothorax, hematothorax and for facilitating image guided operations is considered by the medical practitioners of civilian and military health services counterparts to be beneficial in supporting triage related medical decisions; however, the capability for such non-invasive medical detection system is presently not available to address casualty care support, for example in the field.

It would be advantageous to provide a portable and somewhat automated trauma detection system for use by first responders.

### SUMMARY

In accordance with the invention there is provided a method comprising: providing a 3D image; setting the current resolution to a first coarse resolution; setting the current data to a known current data; iterating an iterative process comprising the steps of: pre-processing the ultrasound image in accordance with the current resolution; providing the pre-processed ultrasound image and the current data to a trained system for extracting therefrom objects of interest at the current resolution, the objects of interest extracted with a likelihood above a known threshold; determining data relating to each extracted object of interest to result in first current data; when the current resolution is a finest resolution, stopping the iterative process; and when the current resolution is other than a finest resolution, setting the current resolution to a finer resolution and returning to the start of the iterative process.

In some embodiments, the 3D image is a medical image of an interior region of a body.

In some embodiments, the 3D image is one of an Ultrasound image, a CT image, an MRI image, and a PETScan image.

In some embodiments, the 3D image comprises an ultrasound image and wherein pre-processing comprises denoising the 3D image to produce a 3D image having a current resolution.

In some embodiments, pre-processing comprises region enhancement of the 3D image to produce a 3D image having a current resolution and with contrast enhancement between regions.

In some embodiments, objects of interest comprise organs of interest and fluid regions.

In some embodiments the method comprises fluid classification of extracted fluid regions.

In some embodiments, determining data relating to each extracted object of interest comprises comparing a likelihood that a first extracted object of interest against a known threshold and when the likelihood is below the known threshold excluding data relating to the first extracted object of interest from the first current data.

In some embodiments, determining data relating to each extracted object of interest comprises comparing a likelihood that a first extracted object of interest is an object of interest against a known threshold and when the likelihood is above the known threshold including data relating to a location and orientation of the first extracted object of interest within the first current data.

In some embodiments, each iteration of the iterative process relies upon a different trained system, the different trained system trained at a resolution appropriate to a current resolution associated with an iteration during which the different trained system is relied upon.

In some embodiments, there are 4 different trained systems.

In some embodiments, the trained systems comprise neural networks other than deep learning neural networks.

In some embodiments, the neural networks rely on region-specific classifiers.

In some embodiments, the neural networks rely on some classifiers that vary with resolution.

In some embodiments, the neural networks rely on some classifiers that remain constant with changes in resolution.

In some embodiments, the trained systems comprise deep learning neural networks.

In some embodiments, the trained systems comprise expert systems.

In some embodiments the method comprises when the iterative process is stopped, identifying potential internal bleeding based on the determined data.

In accordance with another aspect there is provided a computer aided diagnostic system comprising: a plurality of trained software processes, each for operating at a higher resolution to extract from three dimensional image data first data relating to an object of interest, the plurality of trained software systems each trained at a different known resolution and each for receiving iteration data based on results of operation of a previous lower resolution trained software system of the plurality of trained software systems, the iteration data providing approximate location information relating to a detected object of interest.

In some embodiments, each of the plurality of trained software processes is trained for detecting objects of interest and wherein for each detected object of interest of the detected objects of interest, filtering is performed to determine a likelihood that said detected object of interest is an object of interest and when the likelihood is below a threshold likelihood removing said object of interest from the detected objects of interest.

In some embodiments, the plurality of trained software processes comprise neural networks.

In accordance with an embodiment there is provided a method comprising: providing a trainable system comprising a first trainable system and a second other trainable system, each for being provided an initial estimation and for detecting objects of interest within an image; providing training data comprising an image having a known first resolution and first object of interest data indicative of a presence and a location of an object of interest; training the first trainable system based on the first object of interest data; providing a same image having a second resolution finer than the first resolution and second object of interest data indicative of a presence and a location of the object of interest at the second resolution; and training the second trainable system based on the image and the second object of interest data.

In some embodiments, the trainable system comprises a third trainable system and comprising: providing a same image having a third resolution finer than the second resolution and third object of interest data indicative of a presence and a location of the object of interest at the third resolution; and training the third trainable system based on the image and the third object of interest data.

In accordance with an embodiment there is provided a method comprising: providing three-dimensional diagnostic image data having an image resolution; adjusting the resolution of the three-dimensional image to a coarse first resolution; extracting from the three-dimensional image at a coarse first resolution first objects of interest; providing first data relating to the extracted first objects of interest to a second iteration; operating the second iteration on the three-dimensional diagnostic image data at a second resolution finer than the first resolution and extracting second objects of interest from the three-dimensional diagnostic image data at the second resolution, the second objects of interest based on the first objects of interest extracted at the first resolution; providing second data relating to the extracted second objects of interest to a third iteration; operating the third iteration on the three-dimensional diagnostic image data at a third resolution finer than the second resolution and extracting third objects of interest from the three-dimensional diagnostic image data at the third resolution, the third objects of interest based on the second objects of interest extracted at the second resolution; and based on the third objects of interest performing computer aided diagnostics.

### BRIEF DESCRIPTION OF THE DRAWINGS

Exemplary embodiments of the invention will now be described in conjunction with the following drawings, wherein similar reference numerals denote similar elements throughout the several views, in which:

FIG. 1 is a simplified diagram of a models for physical actuator-based ultrasound scanning;

FIG. 2 shows a simplified two-dimensional array representative of a two-dimensional array transducer that is divided into sets of 4 cells, 2×2, allowing for multiplexer addressing of one quarter of the array elements at a time;

FIG. 3 is a simplified architectural diagram of a connector board supporting 32 channels—128 channels when addressed sequentially for four image capture operations;

FIG. 4 is a simplified diagram of a housing including a sensor, processing boards, and interconnects in the form of cabling;

FIG. 5 is a diagram showing illumination patterns achievable with a single firing in some embodiments of the invention;

FIG. 6 is a group of images formed with a four-dimensional (3 spatial and time) ultrasound showing different anatomical features of interest;

FIG. 7(a) is a simplified schematic diagram of an image capture system for capturing a digital image relying on n separate image capture operations to form a single image;

FIG. 7(b) is a simplified schematic diagram of a processing system for processing image data within the frequency domain;

FIG. 7(c) is a simplified schematic diagram of a plurality of processing systems shown in FIG. 7(b) operating in parallel;

FIG. 7(d) is a simplified timing diagram for processing of captured image data;

FIG. 8 is a simplified architectural diagram showing data connections between different components of a parallel architecture ultrasound processing system;

FIG. 9 is a simplified block diagram of a method of automated diagnosis relying on training data;

FIG. 10 is a simplified flow diagram for a method of computer aided diagnosis;

FIG. 11 is a diagram of a geometric shape similar to that of an internal organ;

FIG. 12 is a simplified flow diagram of a method of training a computer aided diagnostic method and system;

FIG. 13 is a further simplified flow diagram of a method of training a computer aided diagnostic method and system;

FIG. 14 is a further simplified flow diagram of a method of training a computer aided diagnostic method and system;

FIG. 15 is a simplified flow diagram of a method of computer aided diagnosis based on a trained classifier;

FIG. 16 is a geometric diagram for describing exemplary extraction of an object from within an image;

FIG. 17 is a simplified flow diagram of an iterative process for extracting objects from an image relying on a previously trained classifier;

FIG. 18 is a simplified diagram of exemplary results of an iterative classification process such as that shown in FIG. 17; and

FIG. 19 is a further simplified diagram of exemplary results of an iterative classification process such as that shown in FIG. 17.

### DETAILED DESCRIPTION OF EMBODIMENTS

The following description is presented to enable a person skilled in the art to make and use the invention and is provided in the context of a particular application and its requirements. Various modifications to the disclosed embodiments will be readily apparent to those skilled in the art, and the general principles defined herein may be applied to other embodiments and applications without departing from the scope of the invention. Thus, the present invention is not intended to be limited to the embodiments disclosed, but is to be accorded the widest scope consistent with the principles and features disclosed herein.

Definitions:

Object under test (OUT): An object under test is any object, objects, and/or body that is being imaged using imaging techniques.

Two-dimensional (2D): Two-dimensional implies that a result is traversable along each of two separate dimensions. Many different two-dimensional coordinate systems are known including Cartesian co-ordinates, radial co-ordinates, etc.

Three-dimensional (3D): Three-dimensional implies that a result is traversable along each of three separate spatial dimensions. Many different three-dimensional coordinate systems are known including Cartesian co-ordinates (along each of three orthogonal vectors), cylindrical co-ordinates, conical coordinates, etc.

Four-dimensional (4D): Four-dimensional implies that a result is traversable along each of three separate spatial dimensions and a temporal dimension—time, providing a three-dimensional image for each of a series of instances in time.

First Coarse Resolution: the first coarse resolution is a resolution coarser than subsequent finer resolutions and is defined herein as the first and coarsest of the resolutions for feature extraction in a set of iterations. The first coarse resolution is an arbitrary resolution more coarse (less information) than a captured image from which it is derived.

Trained System: A trained system is a system that in a first training mode receives training data relating to successful outcomes and uses that training data to form a dataset for use in another normal mode of operation.

No portable ultrasound systems are presently available that provide automated diagnostic tools with ease of use. Further, there is no system that addresses military needs for portable, self contained, field operable equipment at a front line of combat operations.

Available portable ultrasound systems provide two-dimensional images. Unfortunately, two-dimensional images are quite limited in their representation of internal scanned objects and, as such, require a trained medical professional to mentally integrate multiple images to develop a three-dimensional impression of internal scanned objects. Not only does two-dimensional ultrasound imaging require significant training to be useful, but it is also time-consuming and inefficient, making it very poorly suited to use in war zones and at the scene of traumatic events. Making a system that involves less human concentration and less time is important for triage type applications. Making a system that requires less training is important for wider adoption and deployment, as well as for versatility and scalability of system utilization.

Problematically, prior art solutions to 3D volumetric imaging systems are slow and require extensive hardware that is not portable in nature. The resulting systems are ill suited to mobile applications such as for use in triage situations, including those in both civilian settings and in military combat situations. Further, miniaturization of these systems, if possible, would only make them slower and even more poorly suited to battlefield use.

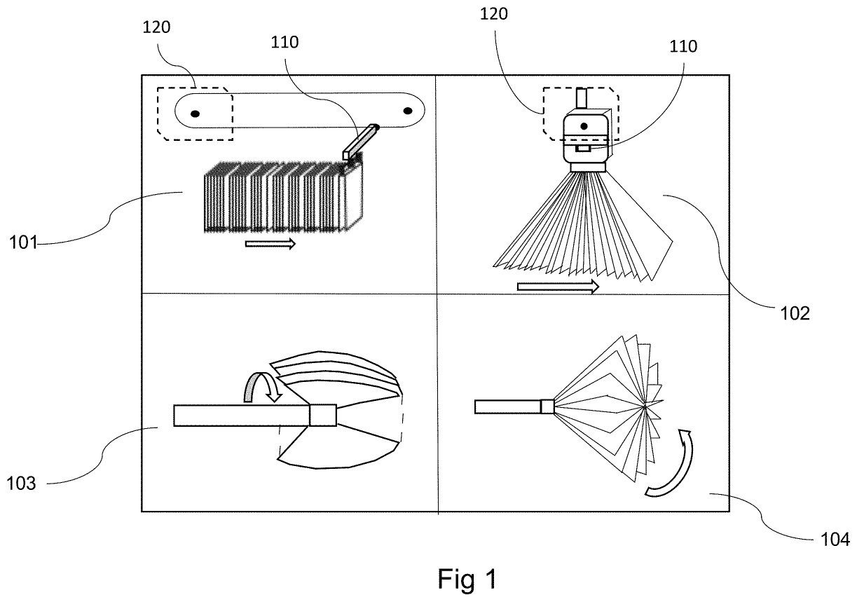

Three-dimensional ultrasound imaging systems have 3 components: an image acquisition circuit, a data reconstruction circuit for constructing a three-dimensional or four dimensional image, and a display circuit for displaying the resulting image data. The three-dimensional image reconstruction process is achievable by a mechanical scanning technique as depicted in FIG. 1. In other solutions, a matrix planar array ultrasound probe is used.

For mechanical scanning, a linear ultrasound transducer 110 is mounted for being moved by a motor 120 as shown in the top two images 101 and 102 of FIG. 1. The motor 120 moves the linear array 110, either laterally across the object under test as shown image 101 or angularly as shown in image 102 to capture the object under test from a single scanning location in a sweeping fashion. The movement of the ultrasound transducer 110 is continuous, cardiac and/or respirator. In addition, the spatial-sampling frequency of the image acquisition is adjustable based on the elevational resolution of the transducer and the depth of the region-of-interest. Thus, for linear scanning, 140 images with a resolution of 336×352 pixels each are collected at 0.5 mm spatial intervals with a time interval dependent upon the ultrasound system frame rate and whether cardiac gating is used. Typically, scanning parameters are adjustable based on requirements of a given experiment or application. For example, for three-dimensional B-mode—typically 2 or 3 focal zones are used resulting in a capture rate of approximately 15 frames/sec and a total three-dimensional imaging scanning time of 9 seconds for 140 ultrasound transducer images. Although this technique does produce useful three-dimensional image data, it suffers from major limitations that preclude its use for general diagnostic procedures. Notably, manual scanning of the three-dimensional space with a linear array does nothing to eliminate false components within the reconstructed B-scan images that are inherent in B-scans.

Three-dimensional image acquisition processes for matrix planar array ultrasound probes, are technically more challenging. Deployed planar arrays include a large number of transducers, typically in the range of 64×64=4096 elements; however, processing and architectural limitations result in the number of elements used being approximately 8×8=64 for a 3D beamformer. Thus, the array gain of a small size sub-array of typically 8×8=64 elements is reduced by approximately: 10×Log 10(64)=18 dBs when compared to the full array gain that would have been provided by the full planar array of 64×64=4096 elements, had it been usable.

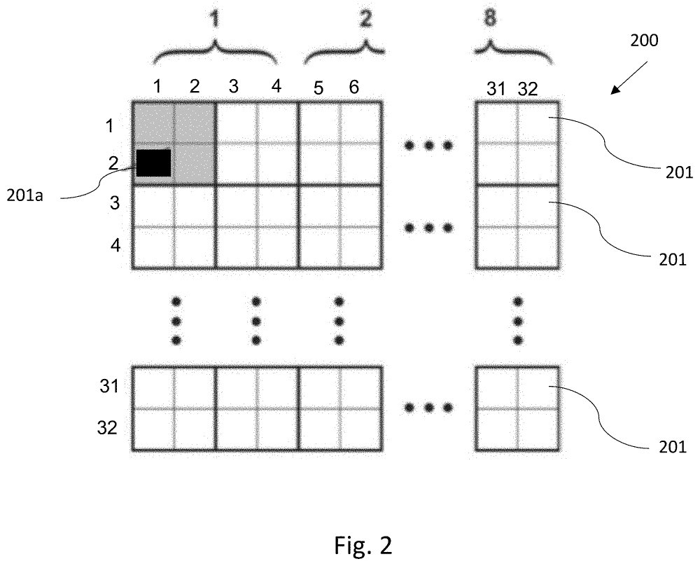

For the sake of simplicity and without any loss of generality, the three-dimensional ultrasound beamformer coherently processes a received signal of only 8×8=64-elements or 4×4 =16 elements, which is a sub-aperture of the 64×64=4096-elements within the ß planar array shown at 200 in FIG. 2. Active transmission takes place approximately every 0.3 ms, depending on desired penetration depth in the object under test. This is much faster than the 4 seconds taken for each image when mechanical scanning is being used.

When an active transmission is completed, the receiving 8×8=64-element sub-aperture is shifted to the left or right by a few elements. Thus, to make use of all the 4096 elements of the deployed probe, the 8×8=64 element beamforming process is repeated at least 32 to 64 times, generating numerous beams at numerous times. For a 4×4=16 sub-aperture, there is at least 4 times as many beamforming processes over the 8×8 aperture resulting in 128-256 imaging operations.

As a result, resulting angular resolution characteristics of reconstructed image data are defined by array gain of the three-dimensional beamformer of the 8×8=64 element sub-aperture as opposed to by the 64×64=4096-element planar array.

Current solutions do not support a mobile, light-weight, easy-to-use, easy-to-operate, trauma detection and assessment system. Instead, significant hardware and complexity is required and due to complexity and performance is ill suited to battlefield and triage applications.

By recording a three dimensional volumetric region with deep penetration (i.e. 24 cm) and wide 3D angle coverage 80°×80°, relevant anatomical structures of interest will be included in the volumetric image and therefore, convenience in locating an anatomical structure and a probability that such a structure is missed by a paramedic with knowledge of human anatomy, will be very low. Moreover, by enhancing image quality and including a temporal component in the sample volume—a time line of successive images, a rapid diagnostic assessment in remote areas becomes more reliable and robust, even without specialized training in system operation.

Alternatively, as shown in 103 and 104, though resulting in similar issues, a rotation of a linear array is used to illuminate and capture image data relating to a three-dimensional volume.

In the present embodiment, the following elements are combined to provide a 4D digital ultrasound system. Of course, for diagnostics, better imaging likely results in better diagnostics, but is not necessary for all CAD results. Therefore, though an advanced 4D ultrasound imaging system is described for the present embodiment other imaging systems are also supported.

A two-dimensional Matrix Array Probe was used in implementing a computer aided diagnostic system according to the present embodiment. The hardware system was developed, relying upon transducers available such as from Fraunhofer-IBMT (St. Ingbert, Germany), which has built a 32×32 transducer 2D planar array probe that has been integrated with an experimental prototype 4D ultrasound system, detailed hereinbelow. Vermon, in France, has also built a multi-dimensional planar array with 32×32 elements. However, a 16×16 or 64×64 planar array is also suitable.

The planar array incorporated the following structures in order to result in substantial miniaturization and improved simplicity of the remaining system architecture.

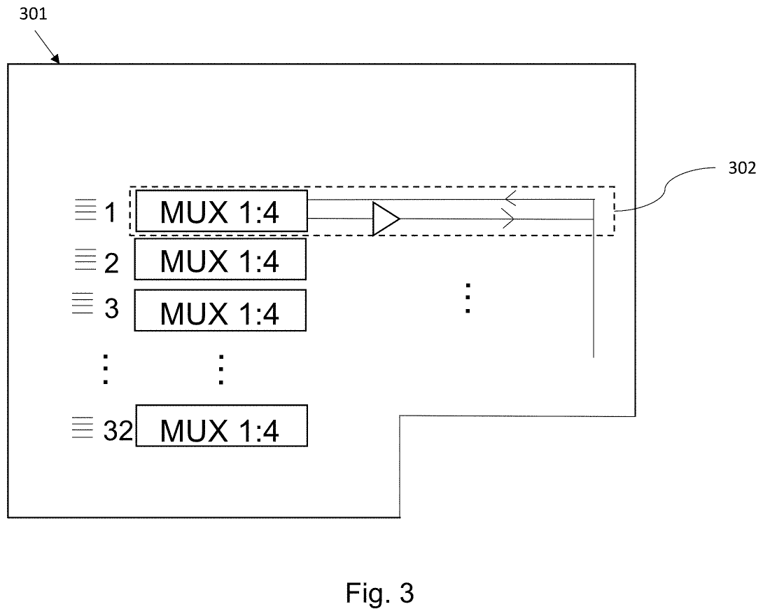

A multiplexer (MUX) for multiplexing planar array elements within the probe. In the preferred embodiment, as shown in FIG. 2, a 4 element MUX-2×2—(not shown) is used to select between four neighboring array elements 201, as shown at (1;1) to (2;2), the four elements forming a single switching group. Each array element comprises a piezoelectric element 201a within a same array and implemented on one connector board designated by numerals 1, 2, . . . 8 as shown in FIG. 3. Thus, though the array of FIG. 2 comprises 32×32 elements, only 16×16 elements are addressed for reading simultaneously via the 4:1 MUX's.

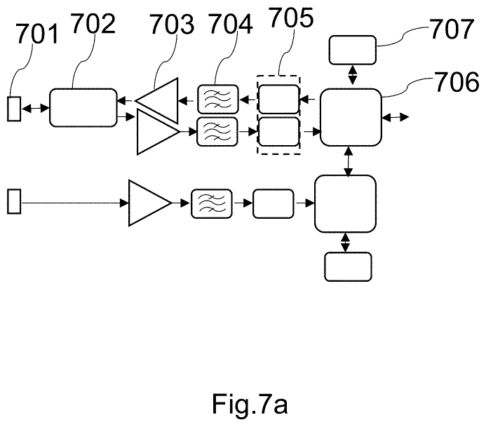

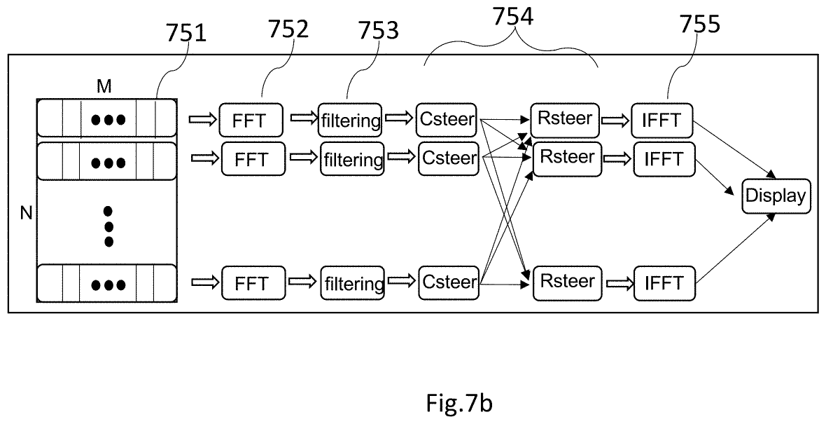

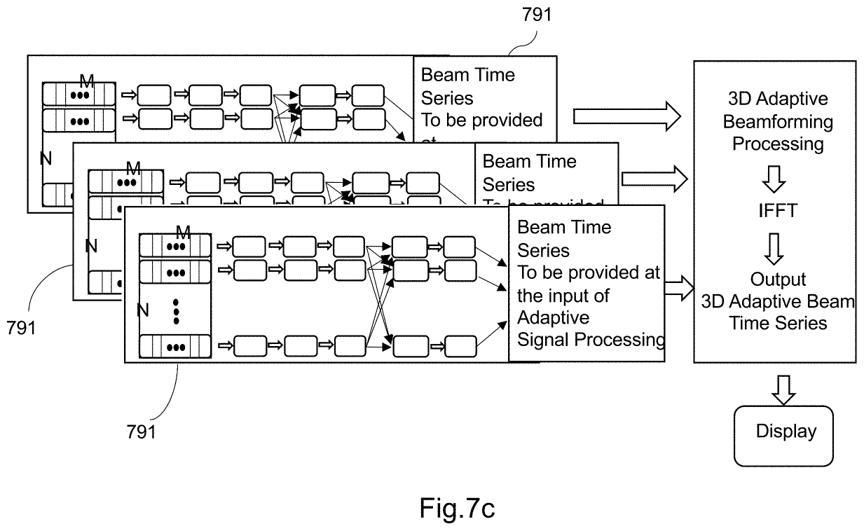

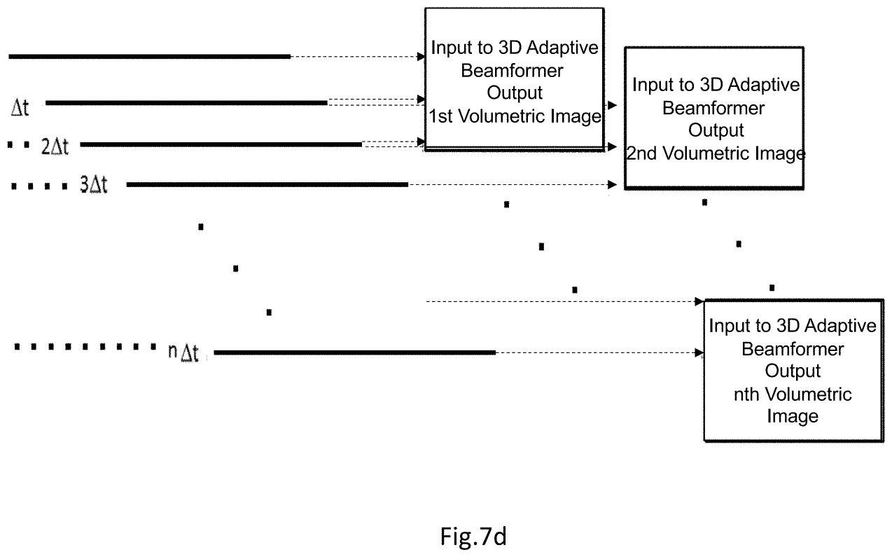

In an exemplary embodiment with a 32×32=1024 planar array, 1024 Analog to Digital Conversion (ADC) operations are performed. Because of the inclusion of MUX for addressing transducers, the ADC operations are performed in four (4) sequential sets of operations thereby relying upon 256 Analog to Digital Conversion (ADC) circuits with each circuit performing 4 Analog to digital conversions in series—one after another. In FIG. 3, a simplified bus diagram for a connector board 301 having 32 multiplexer and Analog to Digital Conversion (ADC) channels 302, thereby supporting 128 channels is shown; 8 such boards are used to support the total number of the 1024 channels of the 32×32 planar array with 4 successive operations of 256 channels. Referring to FIG. 7(a), shown is a simplified block diagram of each channel. A transducer group 701 is coupled via a MUX 702 to the connector board. On the connector board is an amplification circuit 703 for performing pre-amplification of received signals, a filter 704, and analog/digital conversion circuitry 705. A processing circuit in the form of a processor implemented within programmable logic 706 interfaces between the analog/digital conversation circuitry 705 and a memory circuit 707, providing data storage management and processing for the system. Alternatively, another number of ADC circuits is supported on each connector board.

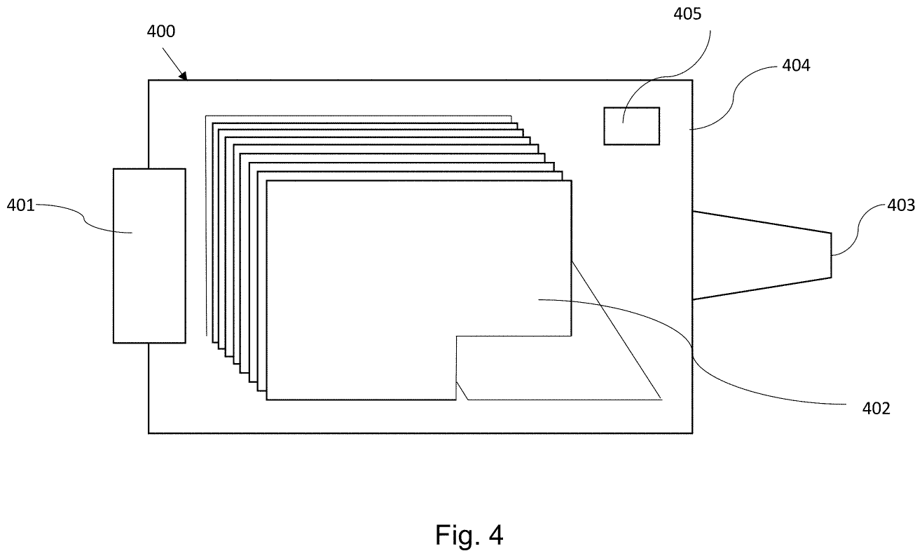

The four (4) 16×16 (256) groups of transducers 701 coupled to each MUX 702, are capable of active transmission of digital active signals through 256-channels of Digital to Analog Conversion (DAC) and reception of acoustic signals through 256-channels of ADC. As a result, pre-amplification functionality provides a protection for the active elements to minimize interference for the remaining reception channels of the matrix array. Referring to FIG. 4, the probe in the form of the sensor head 400, including the planar matrix array 401, is packaged to include connection of the planar matrix array 401 with 8 connector boards 402, backplane (not shown), cabling 403 and housing 404. Thus, the sensor and electronics are housed within a same sensor head 400. A thermal trap or cooling 405 should be included if necessary, as shown in FIG. 4. Alternatively, the sensor is housed in the sensor head 400 and the remaining electronics is housed within a control portion of the system. When this is the case, care should be taken to avoid the effects of noise for each of the 256 channels.

Though the hardware implementation is described with reference to 8 connector boards, and a 4:1 MUX, other configurations are also applicable and optionally depend on the geometry of the planar array. For example, if an 8:1 MUX is used then only 4 connector boards as described hereinabove are connected. Alternatively, each connector board supports only half as many channels and a same number of connector boards are used.

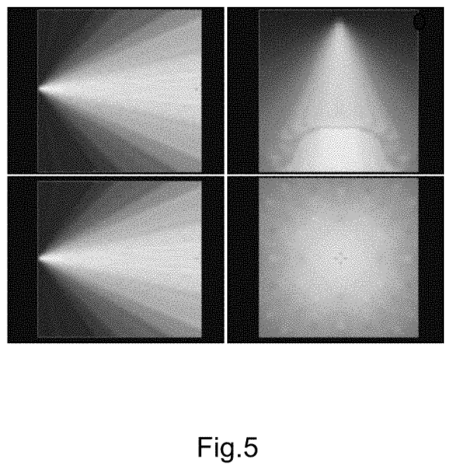

Referring to FIG. 5, the illumination patterns that have been tested, comprise a set of multi-focus, multi-angular sectors that are illuminated simultaneously by a single firing. For comparison, a current ultrasound system utilizes at least 256 firings to illuminate a single plane. The illumination patterns depicted in FIG. 5 simplify an illumination process. This is achieved in the present embodiment through a fully digital design configuration of the illumination driver architecture. Alternatively, another illumination pattern is used, or multiple illumination patters are used within a same system.

The resulting illumination from the above matrix planar array structure is as follows: A conical volumetric segment is imaged with an opening angle of 80 degrees×80 degrees to a maximum depth of 24 cm (sample volume) with an angular resolution of 0.5 degrees and a rate of 20 volumes per second (Vps). A 2D-phased array probe with 32×32 single elements working at a centre frequency of 3.0 MHz is used; the frequency is based on the probe design and with some probes, frequencies such as 7-9MHz are used. All 1024 elements are active during the transmit phase, forming an illumination pattern such as that shown in FIG. 5. During the receive phase, a group of 256 elements are addressed—connected to the 256 electronic channels of the system—by way of addressing the multiplexers. For this embodiment, all multiplexers are coupled to identical addressing circuitry. Alternatively, each multiplexer has its own addressing circuitry and each multiplexer is addressed same during use. Alternatively, the ultrasound signal is designed with different maximum depth and/or different opening angles.

Four (4) receive operations are necessary to acquire ultrasound responses from a whole volume of interest with all 1024 transducer elements. This is done with four (4) transmit operations—transmitting from all transducer elements—each followed by a receive operation—each for receiving information from ¼ of the transducers—wherein addressing of the MUX circuits is incremented between operations. Alternatively, another order of addressing the MUX circuits is also supported.

Switching of the elements between transmit and receive phase and the correct choice of the relevant receive elements is done by addressing and operation of the multiplexers shown in FIGS. 2—4 that are integrated into the probe. The 256 received A-Scans of one shot are digitized with a sampling rate of 25 MHz (or higher), a 14 bit (or 12-bit) resolution, and a length of 4096 samples. After four shots, the 1024 A-Scans of one sample volume are processed by filtering, adaptive beamforming, and scan conversion. The processed A-Scans are then transmitted for display on a monitor, where they are displayed in a 3D representation. This entire process is performed 20 times per second so that a quasi-real-time 4D imaging is achieved. Alternatively, each A-scan results in processing of the A-scan and three previous A-scans to allow for 80 frames per second of quasi-real-time 4D imaging.

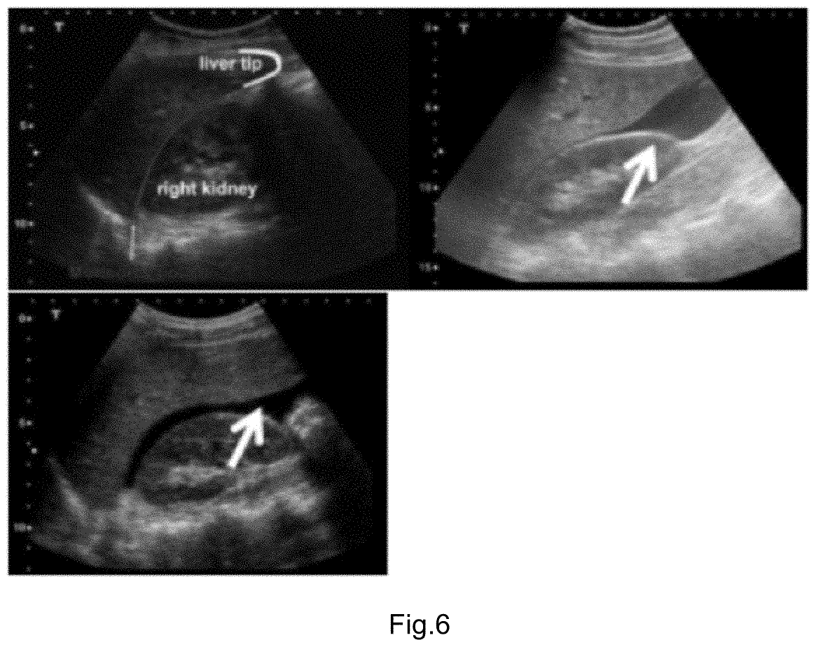

On a monitor is shown an image and a graphical user interface. In the present embodiment, a result of automated detection of free fluid—intraperitoneal free fluid or blood -inside the sample volume is shown in FIG. 6. In exemplary embodiment, automated diagnosis is integrated, but is restricted to detecting free fluid in a characteristic region called Morrison-Pouch, which is the space that separates the liver from the right kidney shown in FIG. 6 and indicated by an arrow. When a person is lying on their back, this is the deepest region inside the abdominal cavity—a region where fluid collects in the case of an internal injury with bleeding. Alternatively, fluid detection is based on a patient position—data entered by the operator. Of course, automatic detection of other objects of interest such as the kidneys, is supported for simplifying system operation.

In U.S. Pat. Nos. 6,719,696 & 6,482,160, a 3D adaptive beamforming method is disclosed. Each of the U.S. Pat. Nos. 6,719,696 & 6,482,160 are incorporated herein by reference. The references taken together define the signal processing structure of an adaptive multidimensional beamformer having instantaneous convergence for ultrasound imaging systems deploying multidimensional sensor arrays. The method provides for enhanced angular resolution of the resulting beamformer. Unfortunately, the method and system are complex and are not amenable to portability and mobile application.

The hardware of the present embodiment shown in simplified diagrams in FIGS. 4 and 7a-7d includes a highly parallelized computing architecture for real-time ultrasound imaging systems deploying 2D and/or 3D multidimensional ultrasound transducer array probes. The probes have planar, cylindrical or spherical geometrical sensor configurations. 3D adaptive signal processing flow and computing architecture layout of the present embodiment are applicable to 3D ultrasound imaging systems deploying either matrix (planar), cylindrical or spherical array ultrasound probes as are known. Alternatively, the probes have other configurations.

Referring to FIGS. 7a, 7b, and 7c, shown are simplified block diagrams of a method to implement 3D ultrasound image capture. Generic decomposition signal processing flow is shown in the diagram. FIG. 7a, as described hereinabove, illustrates the data acquisition unit comprising a set of multiplexers for addressing each of the groups of sensors within the array. Furthermore, the A/DC peripherals are integrated with each one of the 32×32=1024 channels of the matrix (planar) array probe to digitize the transducer signals and to provide the resulting time series at the input of suitably programmed FPGAs to initiate image reconstruction processing. The D/AC peripherals are activated from the FPGAs to illuminate a medium of interest and to trigger a data acquisition process by the A/DC peripherals. The D/AC peripherals are for activating multi-focus illumination patterns, in the form of those shown in FIG. 5. These illumination patterns are unique in a sense that they illuminate the interior of the human body like a flashing light allowing the transducer array to record scattering signals from the illumination and to use the recorded signals within a volumetric image reconstruction process by the 3D Adaptive Beamformer. Referring to FIG. 7c shown is the architecture relying on many processes 791 such as that of FIG. 7b to result in a single image. This allows for quasi-real-time processing of 4D ultrasound image data.

Thus, the architecture of FIGS. 7a, 7b, and 7c for ultrasound imaging systems allows for a coherent processing for a set—in the embodiment {all}—of transducers of a deployed probe. Referring to FIG. 7b, shown is a simplified block diagram supporting the image processing process. Here data relating to an entire image 751 is provided for processing. The data is converted into a frequency domain with a fast Fourier transform (FFT) circuit 752. The converted data is then filtered 753, processed 754 and a reverse FFT is applied at 755 in order to form an image.

As detailed in FIG. 7d, the input information for the 3D adaptive beamformers include beam time series converted to a frequency domain of 3-snapshots from an output port of the conventional—time delay—beamformer as shown in FIG. 7b. A snapshot of data is defined as the planar array time series for supporting a complete volumetric image reconstruction process. This is important for the adaptive beamformer to achieve near instantaneous convergence. Alternatively, longer convergence is supported.

Relying on 3-snapshots by the 3D Adaptive beamformer, to achieve near instantaneous convergence, might be considered as an impediment in that it reduces the rate of the volumetric image reconstruction output images by a factor of 3. The current processing capacity of the computing architecture allows for the reconstruction of 20 volumes/second using the conventional time delay 3D beamformer. Thus the time interval between two snapshots (i.e. two successive volumetric images) is 50.0 ms. As a result, the time interval between two successive volumetric output images of the 3D adaptive beamformer will be 150.0 ms.

This kind of impediment, however, is reduced by introducing, as detailed in FIG. 7d, a re-introduction or re-use of snapshot data to allow for an increase in the 3D Adaptive Beamforming output images to be the same as that of the conventional (time delay) beamformer. Thus, latency remains on the order of hundreds of milliseconds, but every 50 ms a new image is provided at an output port. Thus, and according to the acquisition arrangements of FIG. 7d, the time interval between two successive volumetric output images of the adaptive beamformer is 50.0 ms, except for an initial 100.0 ms of additional delay at the start. Alternatively, with faster processing circuitry, an image is provided at an output port of the beamformer every 12.5 ms with the adaptive beamformer re-using the previous 3 A-scans for each image reconstruction operation—performing one beamforming operation per MUX addressing operation. Alternatively, with enhanced processing speeds, enhanced resolution is supported instead of or in conjunction with increased frame rate.

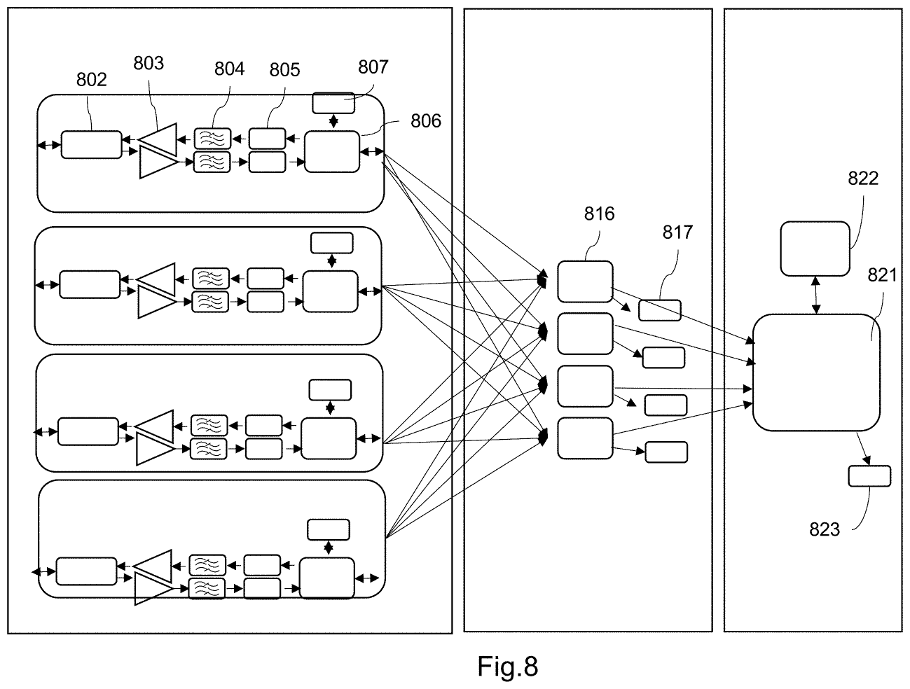

Referring to FIG. 8, shown is a data architecture diagram to highlight data flow during processing. As is evident from FIG. 8, between Csteer operations and Rsteer operations, there is a cross connect resulting in each stage communicating with all parallel subsequent stages. Thus, there is a limit to the amount of parallelization imposed by the communication between stages.

FIG. 8 depicts a highly parallelized fully digital computing architecture for a real time 3D ultrasound imaging system that is capable of deploying a matrix array ultrasound probe with either 32×32=1024-elements or 64×64-4096-elements or 16×16=256-elements.

The design principles of the computing architecture of FIG. 8 reflect the signal processing structure of the 3D Adaptive Beamformer of FIGS. 7c and 7d with elements including MUX 802, amplifier 803, filter 804, DAC/ADC 805, programmable logic 806 and memory 807 present in parallel implementations within the first stage; the first stage includes Csteer operations. In the 2^{nd} stage, programmable logic 816 and memory 817 support Rsteer implementation. In the third stage, a processor 821, a display 822, and a memory 823 allow for visualisation and storage. At the same time, the scalability and generic capabilities, for a variety of medical diagnostic applications, of the proposed computing architecture in FIG. 8, makes it a fully digital real time 3D ultrasound system incorporating unique illumination patterns and a complete digital 3D Adaptive Beamformer with fully coherent array gain incorporating, simultaneously, all transducer-elements of a deployed multidimensional array.

This highly parallelized architecture accommodates processing of from low to highly populated transducer arrays. For example, for a planar array probe with 16×16=256 transducers, the complexity of the computing architecture in FIG. 8, is reduced by a factor of 4 and results in deployment of a smaller number of FPGAs by a factor of 4, when compared to a planar array probe with 32×32=1024 transducers.

Shape-based and atlas-based methods are two known methods for automated organ detection/segmentation in 3D ultrasound images. Both methods present significant drawbacks in real world applications and more so when used in triage or trauma related situations. Shape-based methods rely on an area-based registration—template matching—that finds the maximum matching of the input volume with, for example, the kidney shape model using cross correlation function. After detecting the kidney, an image is segmented using the level-set approach. The drawback of this method is that the area-based registration puts a restriction on detecting kidneys that are deformed by orientation and scaling factors due to the ultrasound probe misalignment. An atlas-based method extracts texture features using Gabor filters. The kidney is divided into a set of sub-volumes, and for each sub-volume, a spatially constraint neural network classifier is trained. This method needs an initial alignment of the kidney shape close to the reference alignment, otherwise the spatially constraint neural network classifiers fail to properly operate. The initialization is performed based on a shape-based method, which has itself some drawbacks. Also, the atlas-based method only uses 3D Gabor filters to extract texture information from input volumetric images, which limits the specificity of extracting discriminative features toward detecting the sub-volume of interest.

Semi-automated or fully automated computer aided diagnosis (CAD) for non-visible abdominal bleeding will enhance usability and in field care for trauma victims. To develop the present embodiment, 3D images have been acquired from a total number of 50 subjects. The data collection started with ascites patients since ascites patients are non-critical cases and can be examined under perfectly controlled conditions, including precise localization of the transducer and a priori knowledge of the amount of injected fluid.

Controlled environment studies cannot be achieved with trauma patients and often-times delays in treatment are simply impossible or have catastrophic results. Some patients present with significant signs of trauma and internal bleeding; however, not all of the trauma patients show critical conditions even when critical conditions are present: in some cases bleeding is rather slow though significant and in other cases visible fluid has other organic origins and is not life-threatening. In such cases after the initial suspicion of inner bleeding, a patient may remain several hours under observation and is repeatedly examined, for example with ultrasound. There is plenty of time to perform 3D ultrasound scans as a part of routine examination of a patient without risking additional harm to patients. These patients are examinable with 2D and 3D FAST protocols.

Inner-body fluids such as blood from inner bleeding typically accumulate at or near the lowest position of the concavities within the body. The site of accumulation of fluid depends on a source of bleeding and the position of a patient. Under normal hospital conditions patients are examined on a bed lying flat on their back. In such conditions inner-body fluids accumulate at the following positions:
Morison's pouch—between the liver and the right kidney—and liver and diaphragm,
Perisplenic / Koller's pouch—along the spleen border,
Suprapubic/Pelvic view—posterior to the bladder—in the male,
the pouch of Douglas/posterial to the uterus in the female, and
Pericardial or suboxiphoid view -pericardical space.

In general, ultrasound refers to high frequency longitudinal mechanical waves, more commonly known as high frequency in the range of MHz sound waves. In theory, ultrasound could propagate in any physical medium, but it has better propagation characteristics when traveling through solid or liquid media that have strong intermolecular mechanical coupling. The intermolecular coupling affects the rate at which the mechanical wave propagates: a solid with strong intermolecular coupling allows for faster ultrasound propagation compared to a low-density fluid with weak coupling. Ultrasound systems have traditionally been used for medical or industrial imaging, mapping out ultrasound reflection points in order to build up an internal image of a target. These systems return information about internal structure of a target but not its composition.

Providing computer aided automated or semi-automated diagnosis using 3D Images from 3D Ultrasound, CT and/or MRI output data as input data facilitates image guided operations in austere front-line environments. For simplicity, computer aided diagnosis (CAD) is used to refer to computer aided automated diagnosis and to computer aided semi-automated diagnosis. It would also allow for better results with less training for ambulance, triage, and large-scale emergency situations. In the present embodiment, a primary diagnostic application is implemented with a 3D Ultrasound System for automated detection of inner-body fluids.

Currently such fluids are detected by scanning the human body on specific characteristic locations by means of the so-called “Focus Assessment with Sonography in Trauma” (FAST) method.

The FAST is an important ultrasound examination method used to identify free intraperitoneal, intrathoracic, or pericardial fluid. Primarily used at a patient's bedside by emergency physicians and trauma surgeons, the development of hand-held ultrasound devices facilitated the introduction of FAST into pre-hospital trauma management (p-FAST).

Clinical indications for a FAST exam are primarily blunt abdominal trauma and penetrating thoracic/abdominal trauma. Despite its low specificity for injury location FAST is a useful screening test for hemoperitoneum. The focused questions of FAST are: 1. Is there free fluid/blood in the abdomen? 2. Is there free fluid/blood in the pericardium?

FAST comprises multiple, focused, ultrasonographic views of an abdomen and a pericardium. The use of multiple views increases sensitivity of the FAST examination in detecting hemoperitoneum. However, FAST as a process requires intensive training and specialization.

An aim of the present embodiment is to minimize training requirements by incorporating a semi-automated or automated diagnostic process within a 3D ultrasound imaging device. Thus, 3D ultrasound data gathering is employed at the corresponding sono-locations and a process automatically analyzes and evaluates a presence of trauma-caused fluids.

An exemplary embodiment of a semi-automated FAST process comprises a plurality of signal processing steps. Volume segmentation is an important part of computer aided medical image analysis. Accurate segmentation results help medical doctors with volume visualization and/or are used for computer-aided diagnosis. Inaccurate segmentation leads to false results and confusion. In the present embodiment, 3D segmentation processes are implemented for kidney, spleen and free fluid, such as blood or water. These segmentation processes are applicable for volumetric 3D ultrasound imaging data.

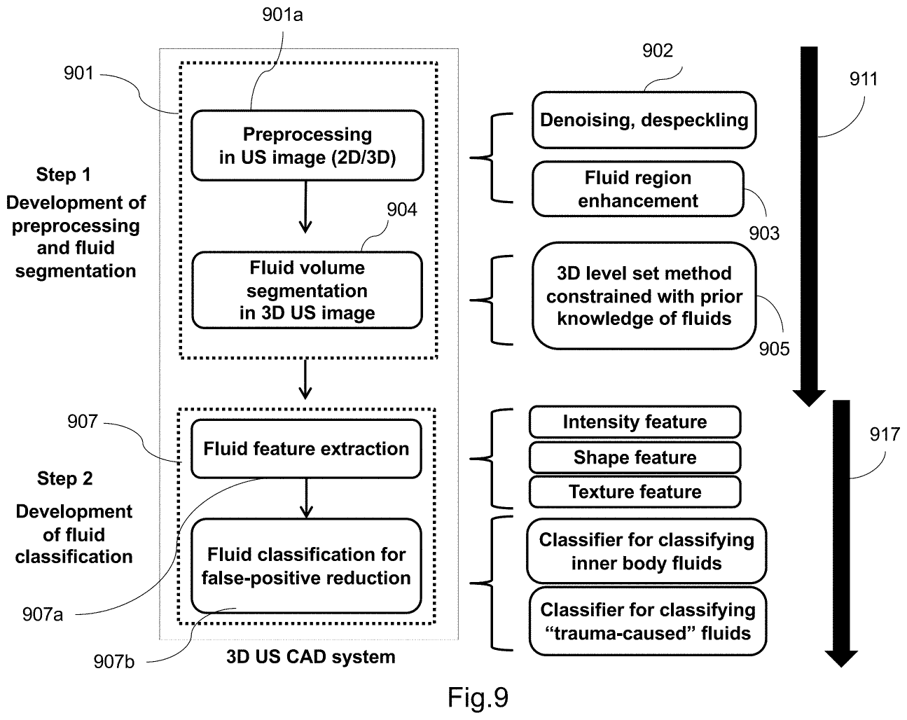

Referring to FIG. 9, in step 1 at 901, pre-processing 901a and segmentation of inner body fluids 904, presented as dark areas in a 3D ultrasound image and including ascites fluid and fluid-containing organs such as vessels, bladder and gallbladder is performed. Though the final target to be detected is the blood fluid presented as dark areas, false positives such as vessels, bladder, gallbladder, and imaging artifacts also exist as dark areas. To differentiate blood fluid from false positives, classifier training with a large amount of training data is relied upon to create a system that accurately distinguishes. Preferably, the system avoids false negatives, as this can be catastrophic, erring instead on false positive outcomes when errors occur. For training, a plurality of 3D Images in each of three views—Morison's pouch view, perisplenic view, and suprapubic view—is relied upon. Training is implemented to train an expert system. Alternatively, the training is implemented to train an Al system such as a neural network.

Segmentation is performed following the steps set out hereinbelow.

At 902, denoising is performed. In ultrasound image processing, denoising is an important process since ultrasound images have significant amounts of speckle noise. Typically, an ultrasound reader such as a doctor, an MD, is trained to ignore the speckle noise and other sources of noise, but it has been found that speckle noise does affect reliability of automated and semi-automated segmentation and, as such, filtering of speckle noise improves system performance for the embodiment described. An effective denoising process is relied upon to support accurate segmentation of inner body fluids. For example, there has been a growing interest in sparse representation of signals. The sparse representation is known to outperform other methods in denoising of Gaussian noise. A sparse representation is sometimes useful for despeckling 2D ultrasound images. In 3D ultrasound image processing, spatial correlation of each slice is also useful for denoising. In the present embodiment, a new sparse representation based denoising framework extended to be suitable for 3D ultrasound image processing and comprising a plurality of steps is as follows:
\textcircled{1} processing speckle noise—multiplicative noise—to result in Gaussian additive noise,
\textcircled{2} Denoising via sparse representation of an ultrasound image, and
\textcircled{3} investigating 3D denoising for accurate segmentation.

At 903 fluid region enhancement is performed to improve segmentation accuracy. Within ultrasound images, fluids appear as dark areas when compared to surrounding tissues. Effectively enhancing bright areas and suppressing darker areas is performed by comparing local statistics—mean and standard deviation—and global statistics. In this step, global and local statistics of a 3D volume are relied upon for enhancing contrast of fluids as follows:
\textcircled{1} Contrast enhancement based on 3D local statistics,
\textcircled{2} measuring local contrast statistics, and
\textcircled{3} enhancing the image to result in a more prominent contrast of fluid.

In segmenting a 3D ultrasound image as shown at 904, fluid suspicious regions are supposed to result in segments. Accurate segmentation is needed because the segmentation results are used for further classification and for visualization—in order to make visualisation meaningful for unskilled system users and accurate. In 2D ultrasound images, an active contour model is widely used for segmentation, because the method shows accurate segmentation results in ultrasound images, even in the presence of strong noise and speckle. Though the active contour model often showed accurate segmentation results, an initial contour to be grown, a seed, should be located close to the actual boundary of a segment. The initial contour is provided by users or prior knowledge (location, size, etc.) of organs in each FAST view in an automatic system. Thus, any existing techniques for segmenting 2D ultrasound images require expert users for accurate results; this is even more pronounced for 3D ultrasound images. In order to simplify a process of segmentation within a 3D ultrasound image, a process is relied upon comprising:
Setting 3D level for a given image at 905;
Providing a numerically stable version of active contour method;
Providing a representation for region merge/separation; and
Active contouring by combining propagation force (region expansion), curvature force (rounding contour), and advection force (locating contour on edge).

An overview of the above processing steps is shown in FIG. 9. This allows for segmentation.

A successful implementation of a computer aided diagnosis software, meets the following two conditions:
1. Task 1: The principal ability and merit of applying 3D ultrasound acquisitions at the FAST locations in order to detect internal bleeding.
2. Task 2: The ability to discriminate trauma-caused fluids from other natural liquids, e.g. blood filled vessels, bladder etc.
   Generally speaking, fluids in ultrasound tend to be darker than tissue. This is true for all types of fluids, e.g. cysts, blood vessels, bladder filling and inner-body fluids caused by ascites or inner bleeding. However, any search has to be oriented towards detection of darker areas.

In the case of bleeding, very fresh blood (say up to 15 min after acute bleeding) tends to appear hyperechoic. However, in the time period after the first 15 min and before 1-2 hours, blood appearance is similar to that of water, i.e. dark and without significant inner structure. After this period however blood begins to coagulate, and inner structure and inhomogeneity become visible as the echo signature becomes more and more hyperechoic. For the scenario contemplated, it is assumed that blood behaves like water during “the golden hour” between casualty and diagnosis.

Cardiac effusion leading to cardiac tamponade is a complex issue. Effusion is somehow more common in car accidents due to the impact between thorax/sternum and driving wheel. However, this is rare in battlefield casualties.

At step 2 at 907, inner-body fluid can be detected in the abdominal or thoracic cavity at 907a and at 907b false positive results are filtered when possible . Blast-caused trauma tends to injure the soft organs located mostly within the abdominal cavity. Thoracic bleedings are frequently caused by various illnesses and are possible also under violent impact injuries, typical in car accidents, however, are rare in blast-caused injuries. A focus of the present embodiment is abdominal bleeding, but other diagnoses are also supported.

Sonographic examination quality may vary depending on both ultrasound equipment quality as well as patient variability (age, fat, previous illness). Older patients and patients with lots of fat tend to degrade the quality of ultrasound, whereas young persons having trained muscles and less or no fat tend to result in better image quality under similar examination conditions. Similarly, the quality of imaging depends on the quality of the ultrasonic equipment used, larger and more expensive equipment gives better image quality than smaller and more inexpensive equipment. Though it is best that examinations are performed with the same hardware used in training, the process herein described was designed to support mid range ultrasound equipment.

Presence of fluid inside the body is not per-se a 100% indicator for injury. Older persons and patients suffering particular diseases such as kidney insufficiency tend to gather visible/detectable amount of fluid inside the body; such a fluid collection is regarded as “normal” or “typical” for their condition and not as sign of trauma or other similar injury. However, for military applications we can safely assume that patients are young and trained, not suffering any severe additional disease. In such a case, detection of fluid inside the body can be seen as a sign of injury caused by blast trauma.

In the particular case where the patient is not examined in a hospital environment (e.g. triage or on-site casualty examination), concentration of fluid in each of the above areas may vary. When a patient's head and thorax are elevated, fluid detected tends to gather around the bladder. When a patient lies on one side, then fluid will be gathered on this side around liver or spleen, accordingly.

In some embodiments, fluid feature extraction 907a includes extracting features based on intensity 907a1, shape 907a2, and texture 907a3. Reducing false positives may be achieved using a classifier for normal bodily fluids 907b1 and a classifier for trauma induced fluids 907b2. Segmentation and classification is performed based on training data 911 and 917, respectively.

It has now been found that a hierarchical three-dimensional (3D) registration approach for detecting organs of interest in medical volumetric images is advantageous. The hierarchical model is formed by dividing a shape of interest of a 3D organ into sub-volumes in a set of resolutions from coarse to fine. This hierarchical modeling is then applied to find global solutions for a volumetric registration problem. As part of a solution, this process distinguishes an organ of interest from other structures and other organs and thereby allows for detection thereof.

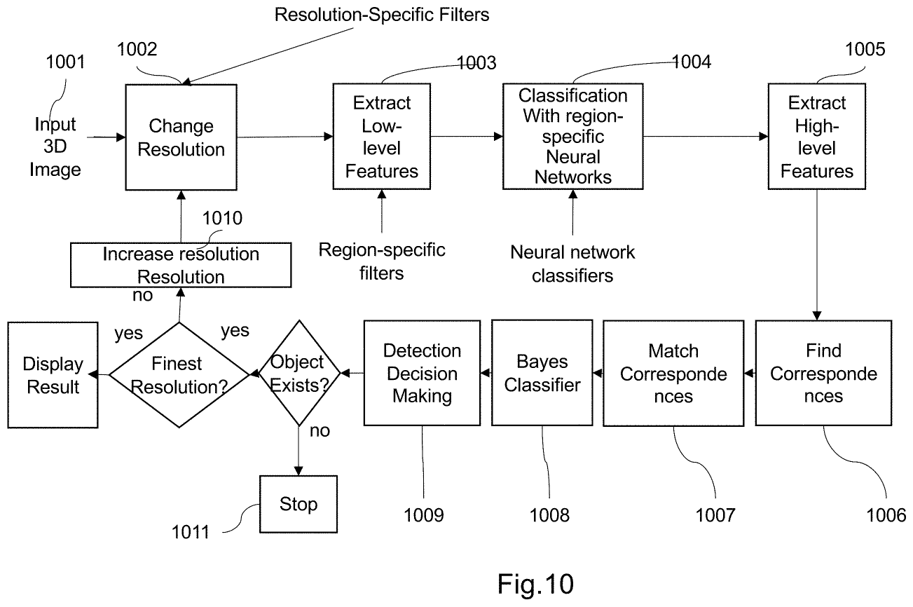

Referring to FIG. 10, the process begins by applying a coarse resolution dataset to a registration process, the coarse resolution dataset representative of a coarsest resolution for the process; when results indicate that a chance of detecting an object of interest is above a predetermined threshold, registration output data is passed to a next iteration of the process at a next finer resolution.

A 3D ultrasound image is provided at 1001. The image resolution is adjusted at 1002. At 1003, low level features are extracted. At 1004, region specific neural networks are used to classify the data to detect organs of interest. At 1005, high level features are extracted and then at 1006 correspondences are found, at 1007 those correspondences are matched, and at 1008 a Bayes classifier is implemented. At 1009 detection decision making is applied to determine whether a likelihood of object recognition having detected an object is sufficiently high to warrant further analysis. When it is not above a pre-set threshold, then the process stops for that object 1011. When it is, the process continues with finer resolutions at 1010, when present.

During each subsequent iteration the process is initiated based on an initial estimation of location and type of object determined from a previous iteration with coarser resolution. Thus, during each iteration, filtering of potential objects is performed in an effort to reduce processing of subsequent iterations and to improve precision and accuracy of results. The process continues with new iterations relying upon finer resolutions as long as during each iteration it is determined that an object of interest—an organ—has a likelihood of existing within the ultrasound image data that is above a predetermined threshold and so long as there exist further classifiers for operating at finer resolution. The probability of accurate detection of an object of interest in the form of an organ within input volumetric image data increases as the process continues to show the object within iterations at finer resolutions. The hierarchical modeling helps to quickly reject false positive cases in early iterations with coarser resolution registration, before iterating into more computationally demanding iterations of finer resolutions.

In building a dataset for iterative application, high-level features are extracted at each resolution. For example, centroid of sub-volumes are high-level features, and each extracted high-level feature in an input image is paired with its corresponding sub-volume's centroid in a reference organ's alignment. Once high-level features are extracted for each resolution, a projective transformation is calculated to map the centroids in an input image to their corresponding centroids within the reference organ's alignment. At each resolution, this process transforms the organ's shape—more accurately defines the organ—to fit better the reference organ's alignment.

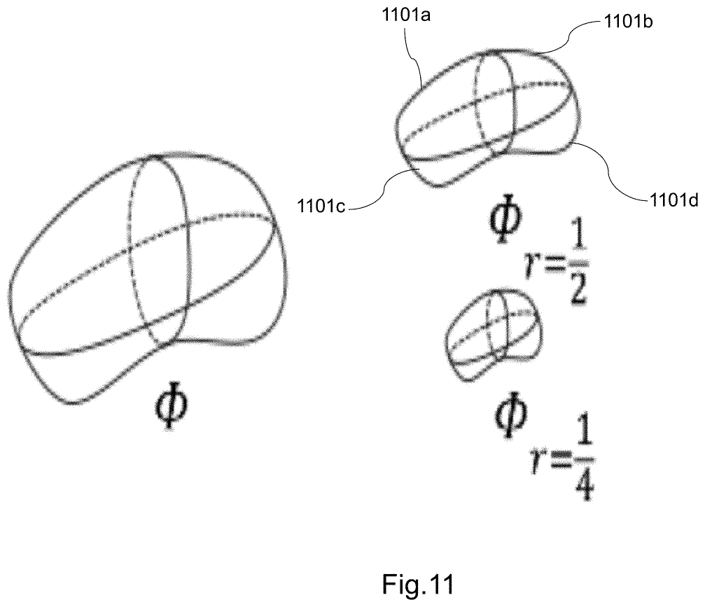

Referring to FIG. 11, the high-level features are extracted by neural network classifiers. Each resolution has a set of trained neural network classifiers, in which each neural network classifier is trained to extract a specific sub-volume 1101a, 1101b, 1101c, and 1101d, respectively of the organ of interest in a specific resolution. Each neural network is spatially constrained to only classify pixels in a specific region of interest (ROI) in the 3D image domain. Starting from coarsest resolution, the ROIs cover the whole entire volume, and going down into finer resolution, each ROI gets smaller and smaller, until at the finest resolution, each neural network's ROI only covers its corresponding sub-volume in the reference alignment of the organ. With such a structural design, the process of hierarchical registration starts with a rough estimation of mapping the organ's shape in the image into the reference alignment. Then it improves the mapping estimation through application of iterations, until at the finest resolution, the organ's shape is expected to fit on the reference alignment.

Thus, in the present embodiment, a plurality of neural networks are trained, each neural network trained for input image data of different resolution and each trained to provide output data that is coarser—more of an approximation—for coarser data and to provide output data that is finer—more precise—for finer data. Thus, 4 neural networks provide a first coarse neural network for determining potential locations of potential objects of interest and the objects' classes, a second neural network for each class for operating on finer resolution image data to extract a likelihood that a detected object and class is accurate and second more accurate characteristics. A third neural network to take output data from the second neural network and to extract a likelihood and third more accurate characteristics. Finally, a fourth neural network accepts as input data a fine resolution image and the output data from the third neural network and performs a final confirmation and localisation on the object of interest based on the input data received. Of course, any number of iterations is supported with 3 or more iterations being preferred.

In the described embodiment, two types of low-level features are used to extract textural information for the plurality of neural network classifiers. Alternatively, other low-level features or more low-level features or different combinations of low-level features are relied upon. Further alternatively, the same low-level features need not be relied upon from iteration to iteration. The two types of low-level features include, region-specific features and resolution-specific features. Region-specific features are sparse learnt-based features, and they are generated using sparse dictionary learning for each sub-volume per resolution. Resolution-specific features are obtained using analytical filters (i.e. 3D Gabor filter), and they sometimes represent, for example, texture information of all regions of the organ of interest. This scheme of low-level features is adopted to allow each neural-network classifier to accurately and flexibly extract voxels to corresponding regions from background. Of course, some low level features such as texture may differ at different resolutions. Other low level features such as darkness remain constant across resolutions.

Since region-specific features are generated from training data for each sub-volume, the neural network classifier is highly discriminative between member and non-member voxels of a corresponding sub-volume. In the case of ultrasound volumetric data, the feature space is highly sparse, and adopting sparse learnt-based features facilitates capturing high textural profile using only a small training data. This is in contrast to deep learning, which demands huge datasets for creating training filters. Alternatively, deep learning is employed to generate the region-specific features. Further alternatively, another form of expert system is trained in place of a neural network.

Once registration is performed with a neural network having a given resolution, a Bayes classifier is applied to validate that a registered shape is actually the organ shape of interest. If the Bayes classifier validates the organ's shape, the registration result is passed to the next iteration at a next, finer resolution, and the iterative process continues. But if the Bayes classifier does not validate that the organ shape exists, the iterations relating to that object of interest and class stop; the branch of the iterative tree is pruned as a likelihood of the object of interest being present is too low.

The present embodiment contains both training phase and run-time designs. Training phase is for (a) generating sub-volumes of each resolution, (b) creating region-specific low-level features for each sub-volume/resolution, (c) training neural network classifiers, and (d) forming shape models of sub-volumes. During a run-time process, training data are utilized to detect within information in input volumetric images, aiming to detect objects of interest in the form of organs and to find a registration that fits the organ's shape with a reference alignment.

In the proposed hierarchical model, the organ of interest is resampled into its lower resolution forms as shown in FIG. 11,

$$\{ r \vert r ∈ [ \frac{1}{{2}^{{N}_{r}}} , \frac{1}{{2}^{{N}_{r} - 1}} , … , \frac{1}{2} , 1 ] \} .$$

At each resolution, an organ is spatially divided into a set of regions. In training phase, a hierarchical model is applied to train filters and classifiers. In run-time phase, the hierarchical model is applied to detect organs of interest and to distinguish same from other internal structures.

For a training phase, considerations account for establishing a successful training result. Suppose a set of training volumetric images is given, {V^{tr}^{ᵢ}|i∈[1, . . . , N^{tr}]}, in which each volumetric image has the same size, V^{tr}^{ᵢ}∈Nₓ×N_{y}×N_{z} . For each training image, V^{tr}^{ᵢ}, a volumetric ground truth data, Φ^{tr}^{ᵢ}, is manually generated that has the same size as its pair volumetric image, Φ^{tr}^{ᵢ}∈Nₓ×N_{y}×N_{z}. Each point in a ground truth, Φ^{tr}^{ᵢ}(x, y, z), specifies whether its corresponding point in the training image, V^{tr}^{ᵢ}(x, y, z), belongs to the desired organ (Φ^{tr}^{ᵢ}(z, y, z)=1), or not (Φ^{tr}^{ᵢ}(x, y, z)=0). A training image is randomly selected as a reference volume, {V^{ref}, Φ^{ref}}. The set of training volumes are used for the following purposes:
training region-specific classifiers to extract high-level features for registration, generating region-specific learnt-based filters to extract features that maximize discriminative power of region-specific classifiers,
generating a set of correspondences from the reference object, and
creating probabilistic shape models to maximize discriminability power of detecting object from non-object using MAP.

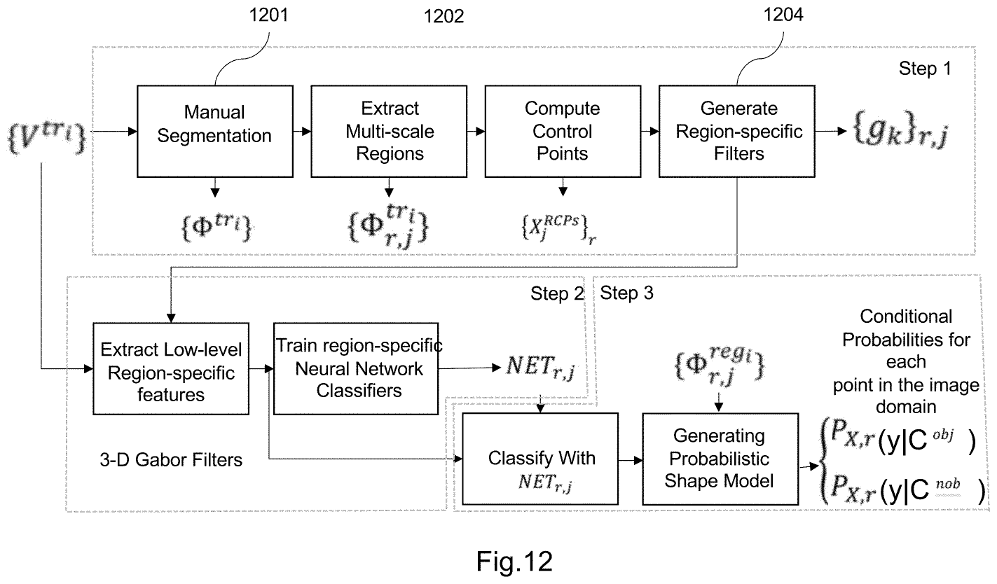

Referring to FIGS. 12, at 1201 and 1202 the organ's shape (specified as {Φ^{tr}^{ᵢ}=1} or simply Φ^{tr}^{ᵢ}) is split into multi-resolutional regions, {Φ_{r,j}^{tr}^{ᵢ}}, where j is the sub-volume division in resolution, r. An example is shown in FIG. 11. Typical organ shape is split into sub-volumes in three resolutions, {r★r∈{1,0.5,0.25}}.

Regions are first specified as multi-resolution divisions in the reference objects, {Φ_{r,j}^{ref}}. It is important to correctly delineate multi-resolution regions in training objects, according to the specified regions in the reference object. Otherwise, it results in a huge training error. Thus, the training volumes are first transformed into the reference coordinate, {Φ^{reg}^{ᵢ}}, and then, their multi-resolutional regions, {Φ_{r,j}^{reg}^{ᵢ}}, are outlined according to the reference object regions. Finally, the multi-resolutional regions are transformed back into their original space domain, {Φ_{r,j}^{tri}^{ᵢ}}. The process of finding transformations to register training volumes on the reference alignment relies upon a human operator to provide input data. First, a set of landmarks are manually specified on the reference image. Then, for each training image, corresponding landmarks are manually selected on the training image. Afterwards, an affine transformation is calculated which maps the corresponding landmarks to the reference ones.

For each region/resolution, centroid is computed using the following equation:

$$\frac{{\sum }_{i ∈ Tr} \frac{{\sum }_{x , y , z ∈ Ω} x {Φ}_{r , j}^{{reg}_{i}} ( x , y , z )}{{\sum }_{x , y , z ∈ Ω} {Φ}_{r , j}^{{reg}_{i}} ( x , y , z )}}{{N}_{tr}} \\ {\{ {X}_{j}^{RCPs} \}}_{r} = \frac{{\sum }_{i ∈ Tr} \frac{{\sum }_{x , y , z ∈ Ω} y {Φ}_{r , j}^{{reg}_{i}} ( x , y , z )}{{\sum }_{x , y , z ∈ Ω} {Φ}_{r , j}^{{reg}_{i}} ( x , y , z )}}{{N}_{tr}} \\ \frac{{\sum }_{i ∈ Tr} \frac{{\sum }_{x , y , z ∈ Ω} z {Φ}_{r , j}^{{reg}_{i}} ( x , y , z )}{{\sum }_{x , y , z ∈ Ω} {Φ}_{r , j}^{{reg}_{i}} ( x , y , z )}}{{N}_{tr}} \qquad ( 1 )$$

The present embodiment relies upon learnt-based filters to increase separability power between voxels belonging to different regions. This characteristic is important for discriminative feature extraction for successful hierarchical registration. Therefore, these filters are called learnt-based region-specific filters.

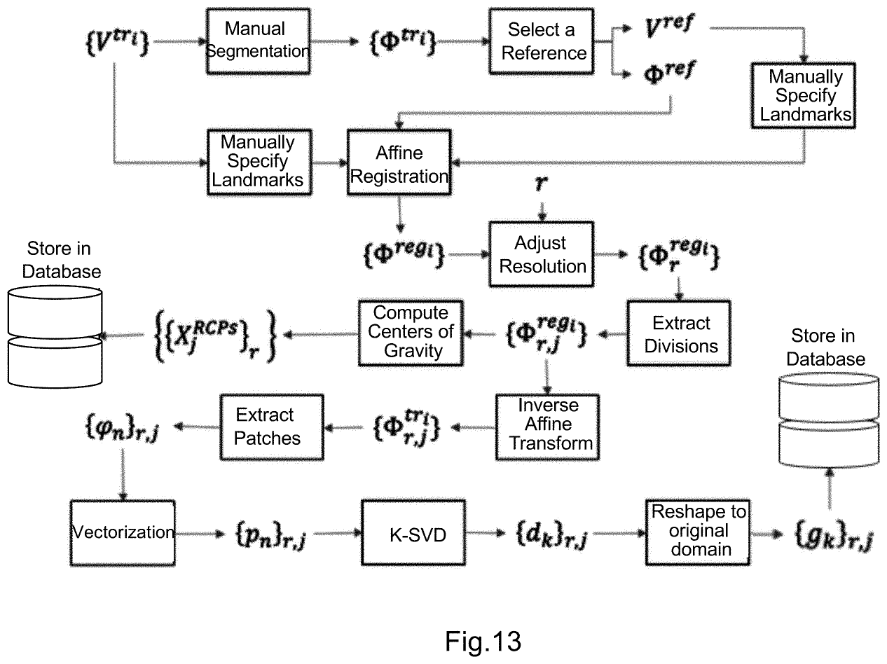

Sparse dictionary learning based on label-consistent discriminative K-SVD is used to train filters for all regions and at each resolution. For each resolution, three-dimensional patches of all the regions are extracted from the training volumes, {φₙ}_{r,j}^{tr}^{ᵢ}. All patches at each resolution are the same size, which is smaller than the smallest region at the resolution. Then, the three-dimensional patches are vectorized {pₙ}_{r,j}^{tris i}, and used to train discriminative dictionaries for the regions, {D_{r,j}|D_{r,j}=[d₁, . . . , d_{N}_{_{Dr}}]}, where dₖ is called a dictionary atom, and N_{D}_{ᵣ} is the number of atoms per each region's dictionary at the resolution, r. Once the dictionaries of all regions of all resolutions are trained, their atoms are reshaped into the original three-dimensional format, and at 1204 the learnt-based region-specific filters are generated {gₖ}_{r,j}. A diagram of this process is shown in FIG. 13.

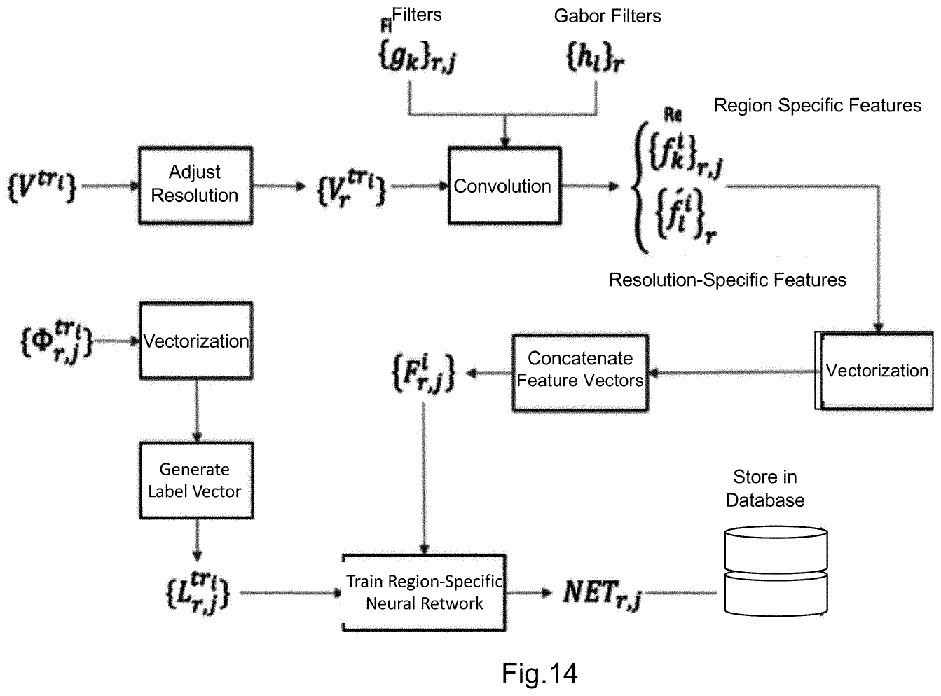

Referring to FIG. 14, according to a method an individual neural network classifier is assigned and trained to classify (separate) voxels belonging to its corresponding region from other regions and non-relevant structures at each resolution. Input data of each region-specific classifier includes (a) features obtained by applying the corresponding learnt-based region-specific filters, {fₖⁱ}_{r,j}, and (b) features extracted by applying resolution-specific Gabor filters, {fₗⁱ}ᵣ. The mixture of resolution-specific and region-specific features provides enough information to train region-specific classifiers to discriminate (a) organ from non-organ regions, and (b) organ regions from each other. The resolution-specific features are calculated once for all region-specific classifiers in a resolution, whereas, region-specific features are calculated separately for each region-specific classifier.

The extracted region-specific and resolution-specific features are vectorized before being provided to the classifiers. Then, both types of features are concatenated to form a region-specific feature matrix, F_{r,j}ⁱ. For each region/resolution, training labels are generated by vectorizing a corresponding ground truth, Φ_{r,j}^{tr}^{ᵢ}, and after concatenation, the matrix of labels for each region is obtained, L_{r,j}^{tr}^{ᵢ}. Once, the region-specific feature and label matrices are created, they are provided to a neural network trainer. This process is applied for all regions in all resolutions, and the trained neural network classifiers are obtained, {NET}_{r,j}.

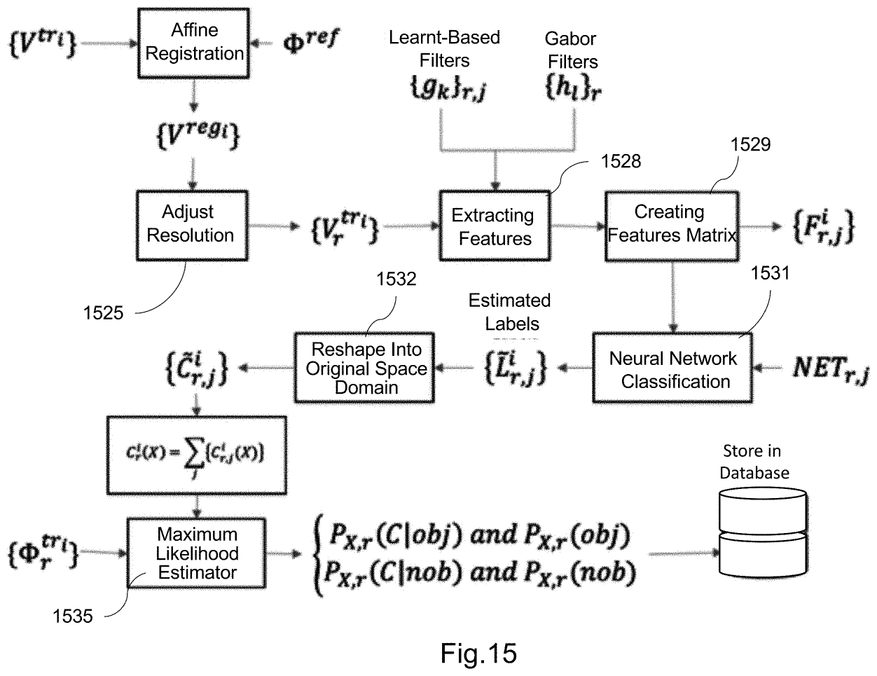

Referring to FIG. 15, a probabilistic shape model is generated to cover morphological variability of the organ's shape of interest. Such a shape model represents probability of each voxel being a member of a given organ of interest. Training volumes are used to estimate a probabilistic shape model. The global deformations from the reference volume are first removed from the training volumes. Thus, instead of using {V^{tr}^{ᵢ}}, their registered versions are applied, {V^{reg}^{ᵢ}}. Then, all the registered volumes are resampled into the resolutions at 1525,

$$\{ {V}_{r}^{{tr}_{i}} \vert r ∈ [ \frac{1}{{2}^{{N}_{r}}} , \frac{1}{{2}^{{N}_{r} - 1}} , … , \frac{1}{2} , 1 ] \} .$$

Afterwards at 1528, the region-specific and resolution-specific filters are applied to extract features from {Vᵣ^{tr}^{ᵢ}}, and after vectorization and concatenation, the feature matrix is formed at 1529. At 1531 the trained classifiers are applied to estimate labels, {\tilde{L}_{r,j}ⁱ}, and then, at 1532 estimated labels are reshaped into three-dimensional space, {\tilde{C}_{r,j}ⁱ}.

The ground truth data of each region-resolution, {Φ_{r,j}^{tr}^{ᵢ}}, is used as prior knowledge to estimate conditional probability model at 1535—Gaussian distribution parameters, and prior probability of being organ or non-organ for each voxel in the three-dimensional image domain. The conditional probability model is estimated using the maximum likelihood estimator (ML). The output data from this process is conditional probability data for each voxel, X̅, belonging to organ and non-organ, P_{X,r}(C|obj) and P_{X,r}(C|nob) , respectively, and Prior probabilities P_{X,r}(obj) and P_{X,r}(nob). The prior probabilities are calculated as

$${P}_{X , r} ( obj ) = \frac{{N}_{obj , X , r}}{{N}_{nob , X , r} + {N}_{obj , X , r}} and {P}_{X , r} ( n o b ) = \frac{{N}_{nob , X , r}}{{N}_{nob , X , r} + {N}_{obj , X , r}} .$$

Once training is sufficiently completed, the trained system is functional for run-time application to perform computer aided diagnosis. Similar processes are relied upon during the run-time phase, wherein organs of interest are extracted to distinguish them from free fluid within a body cavity. During run-time, a first objective is to determine whether an organ of interest exists in an input volumetric image, and if it exists, to determine its alignment in the image. The alignment is represented as a transformation from a reference alignment of the organ in the image domain. Alternatively, the alignment is represented in another fashion. The transformation is obtained through a registration process. Therefore, the run-time phase uses the trained region-specific filters and classifiers, regional centers of gravity, and the probabilistic shape model to register input images on the reference organ's alignment. This is a three-dimensional volume to shape registration process. The process starts at the coarsest resolution, first iteration, and proceeds into the finer resolutions with subsequent iterations, when an object is detected at the coarser resolution.

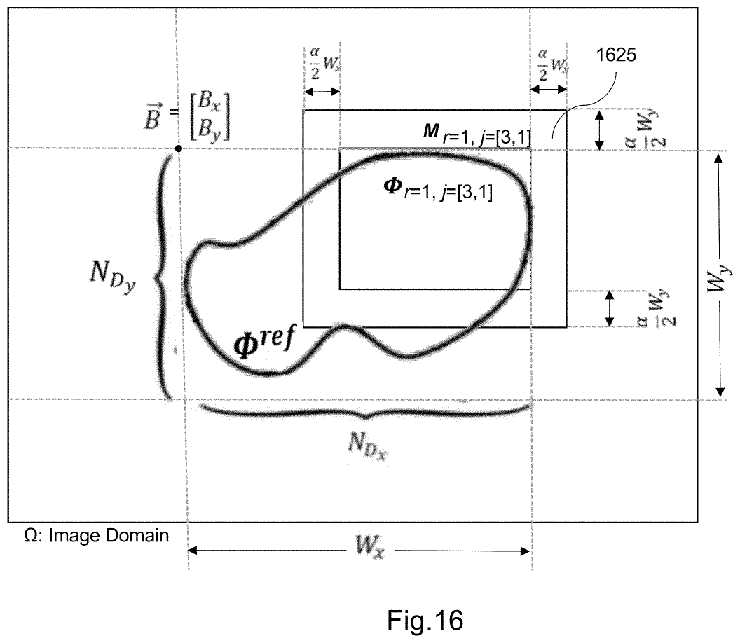

Referring to FIG. 16, a region-specific mask, M_{r,j}∈Nₓ×N_{y}×N_{z}, specifies a sub-volume (box) in the image where the corresponding region-specific classifier, NET_{r,j}, is applied to it. In the coarsest resolution, each masked sub-volume, Mᵣ_{ₘᵢₙ}_{,j}, covers an entire image, to generalize a process of searching for high-level features. As iterations proceed to finer resolutions, each masked sub-volume, M_{r,j}, covers a smaller portion of the image domain aiming to localize searching for high-level features. FIG. 16 shows a 2-D mask, M_{r=1,j=[3,1]} that corresponds to region, Φ_{r=1,j=[3,1}] and activates points inside the rectangle shown 1625.

The coordinates of points specifying the activation box (3-D) in each M_{r,j=[n}_{ₓ}_{,n}_{_{y}}_{,n}_{_{z}}] where nₓÅ[1, . . . ,N_{D}_{ₓ}], n_{y}∈[1, . . . ,N_{D}_{_{y}}] and n_{z}∈[1, . . . , N_{D}_{_{z}}] specify a division, are calculated as follows,

$${x}_{{M}_{r , j}} = r [ {B}_{x} + ( \frac{{n}_{x} 1}{{N}_{{D}_{x}} + 1} α ) {W}_{x} {B}_{x} + ( \frac{{n}_{x} + 1}{{N}_{{D}_{x}} + 1} + α ) {W}_{x} ] \\ {y}_{{M}_{r , j}} = r [ {B}_{y} + ( \frac{{n}_{y} 1}{{N}_{{D}_{y}} + 1} α ) {W}_{y} {B}_{y} + ( \frac{{n}_{y} + 1}{{N}_{{D}_{y}} + 1} + α ) {W}_{y} ] \\ {z}_{{M}_{r , j}} = r [ {B}_{z} + ( \frac{{n}_{z} 1}{{N}_{{D}_{z}} + 1} α ) {W}_{z} {B}_{z} + ( \frac{{n}_{z} + 1}{{N}_{{D}_{z}} + 1} + α ) {W}_{z} ] \qquad ( 2 )$$

where [Bₓ B_{y} B_{z}] and [Wₓ W_{y} W_{z}] specify the coordinate of the top-left corner and size of the box encompassing the reference object Φ^{ref}. α is a searching generality controller coefficient and αᵣ_{ₘᵢₙ}=∞, and αᵣ₌₁=0.

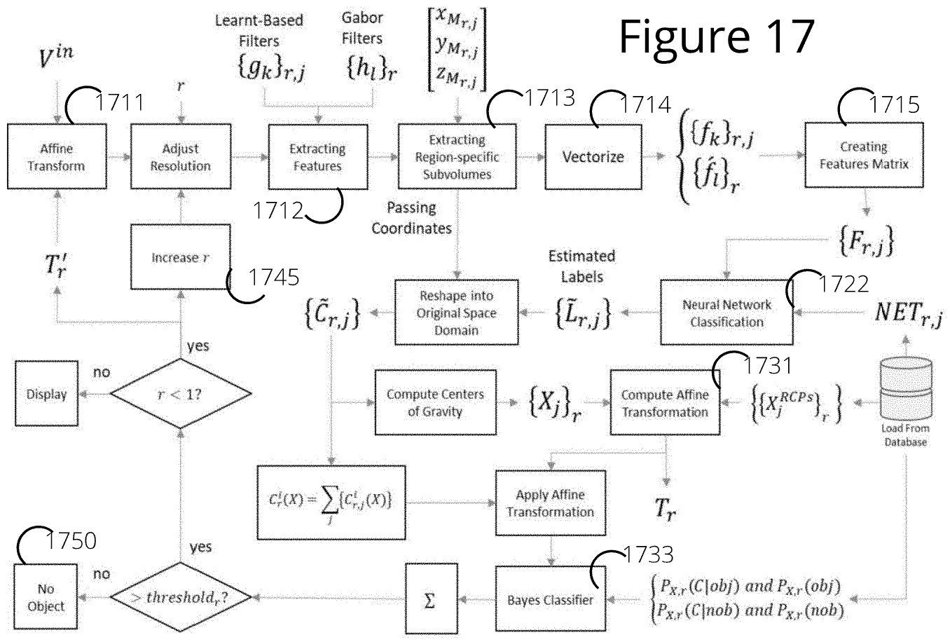

The steps of the registration process shown in simplified block diagram of FIG. 17 are set out below:

Reset resolution to coarsest resolution, r=r_{,in}, and initialize affine transformation matrix, Tᵣ, equal to an identity matrix.

At 1711, resample input volume to the resolution, r.

At 1712, 1713, 1714, and 1715, apply region-specific and resolution-specific features of the resolution, r, on each region-specific mask, M_{r,j}.

At 1722, apply region-specific classifiers to generate high-level features (centroid of regions).

At 1731, calculate an affine transformation that maps high-level features to their corresponding centroids of the reference organ's shape.

At 1732, apply the affine transformation on the volumetric image to obtain the registered volume.

At 1733, applying Bayes Classifier on the registered volume.

When a probability of having an organ present within volumetric data is higher than a threshold value at 1745, provide data relating to that organ to a next iteration at a finer resolution, rₙₑₓₜ=2 r, and go to the step of resampling in the next iteration at the finer resolution. Otherwise at 1750, stop the hierarchical process for that potential organ, and return a rejection flag.

The trained probabilistic shape models are used to determine the probability of having the organ's shape inside the volumetric image at resolution, r. The Bayes classifier is applied to classify voxels into organ and non-organ classes. For each voxel in the registered volume at the resolution, r, the summation of all region-specific neural network classifiers is calculated as, Cᵣ(X)=Σⱼ{C_{r,j}(X)}. Then, for each voxel, its label is estimated using the Bayes classifier as follows,

$${l}_{r} ( X ) = {max}_{{l}_{X}} \{ \begin{matrix}{P}_{X , r} ( {C}_{r} ( X ) \vert obj ; {\tilde{μ}}_{obj} , {\tilde{σ}}_{obj} ) {P}_{X , r} ( obj ) \\ {P}_{X , r} ( {C}_{r} ( X ) \vert nob ; {\tilde{μ}}_{nob} , {\tilde{σ}}_{nob} ) {P}_{X , r} ( nob )\end{matrix} \} . \qquad ( 3 )$$

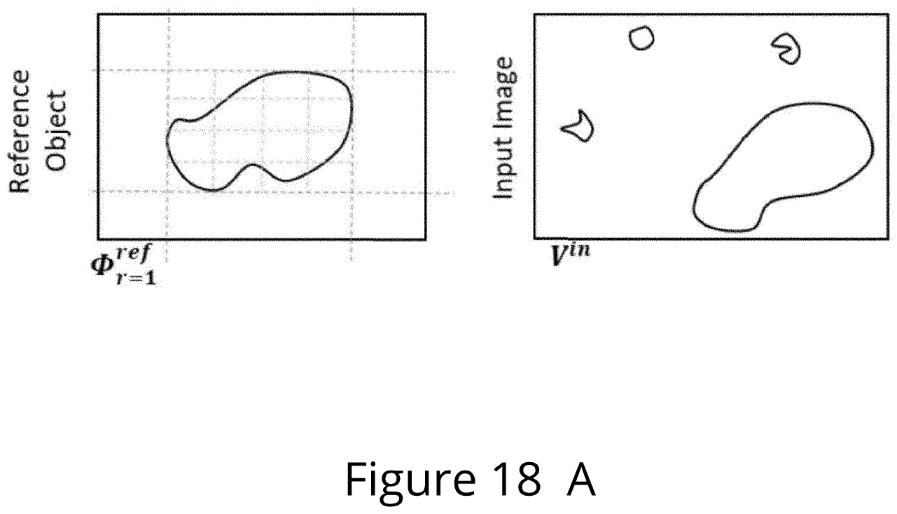

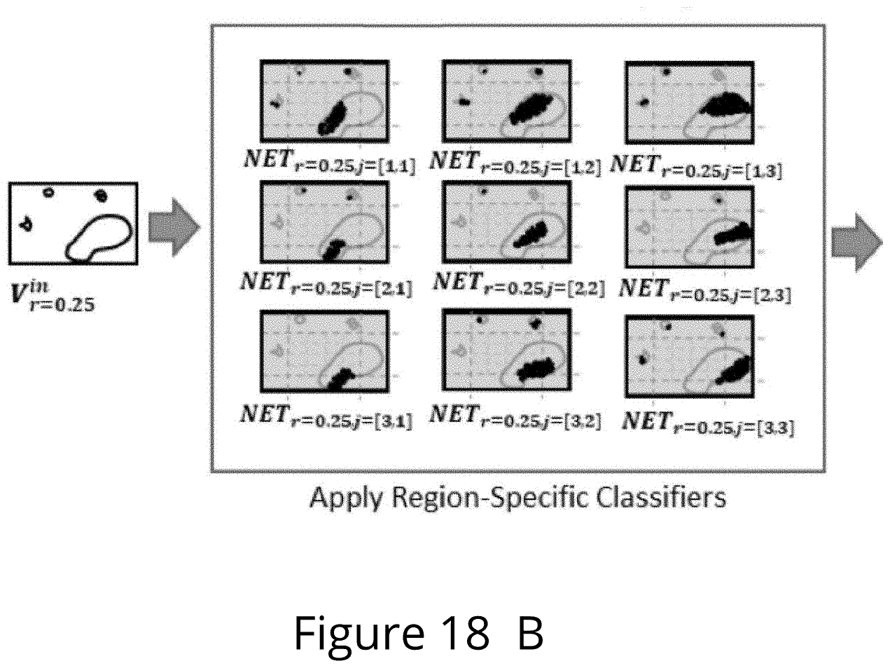

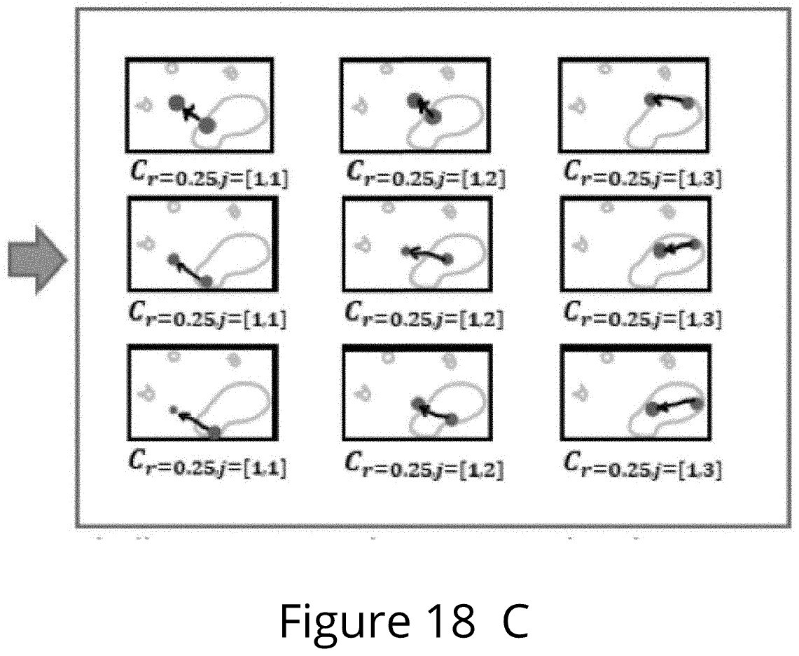

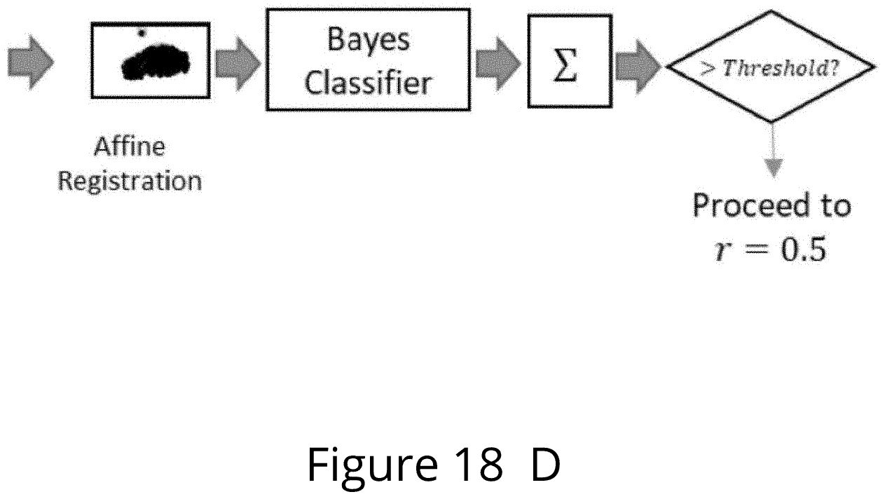

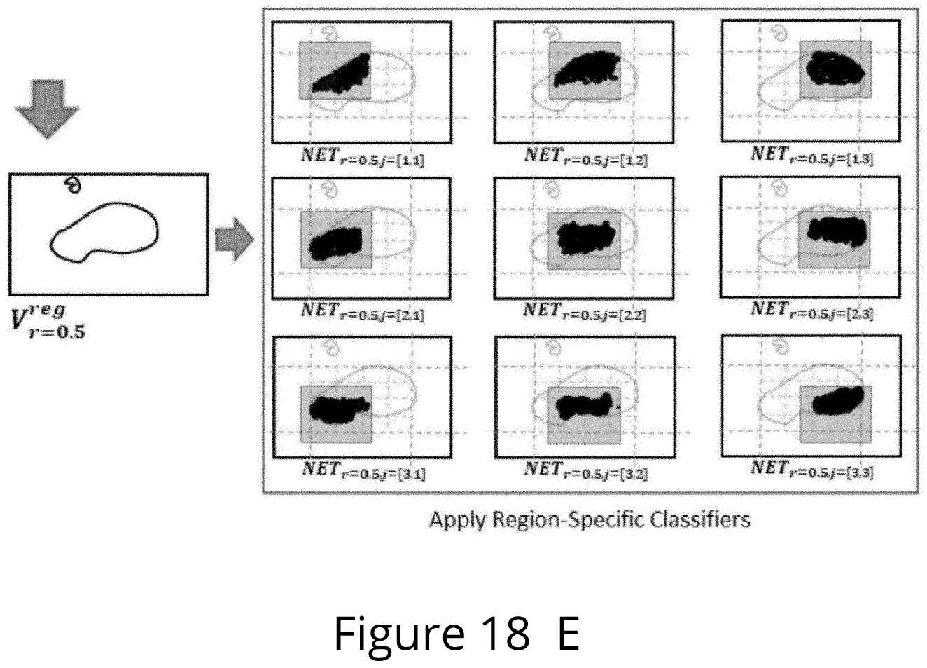

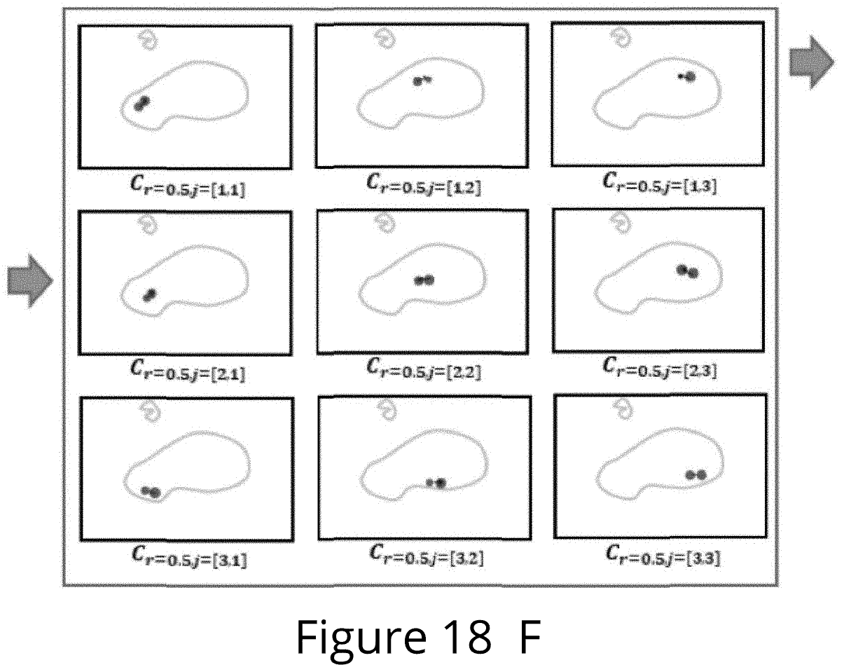

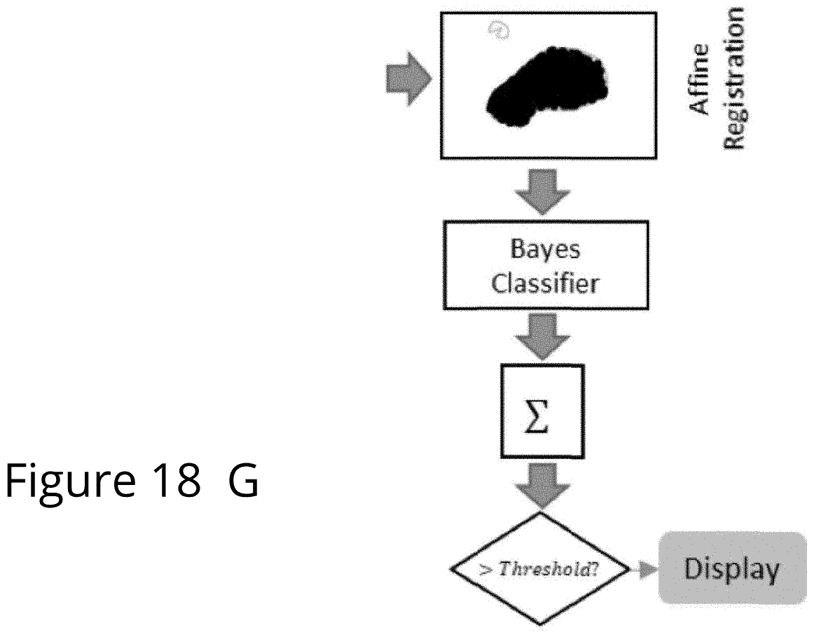

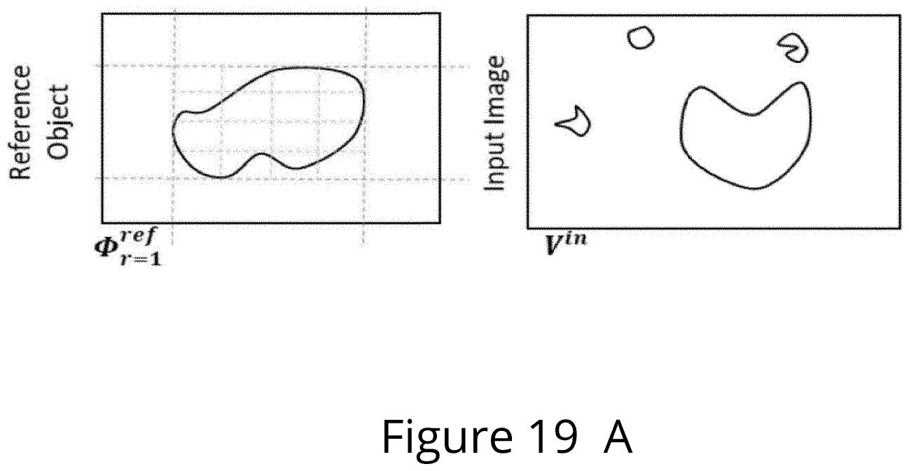

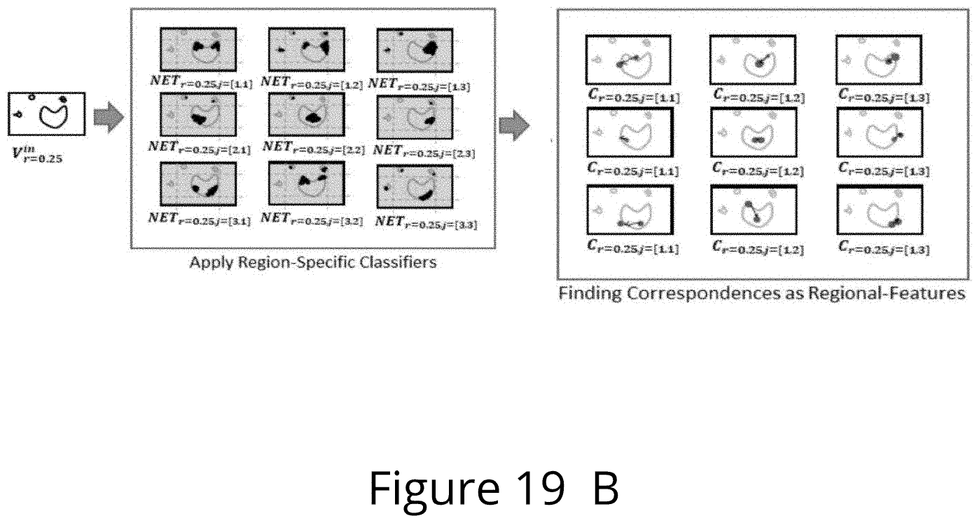

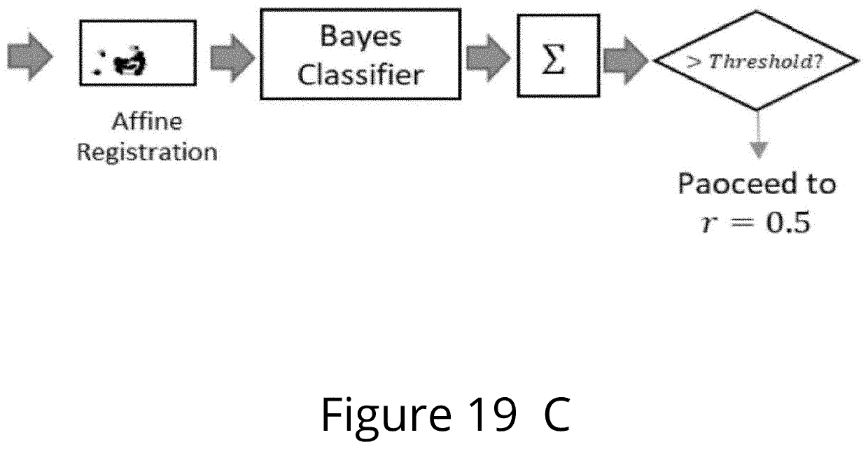

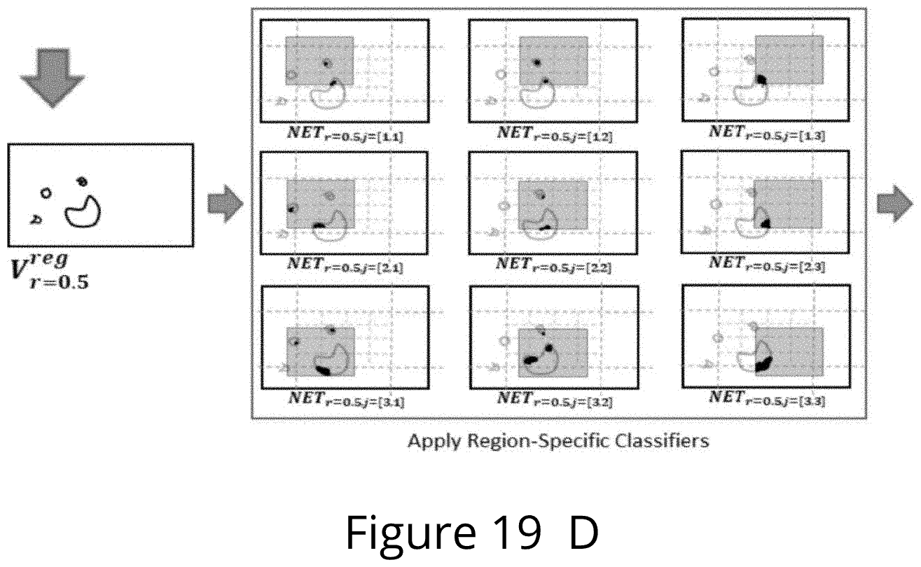

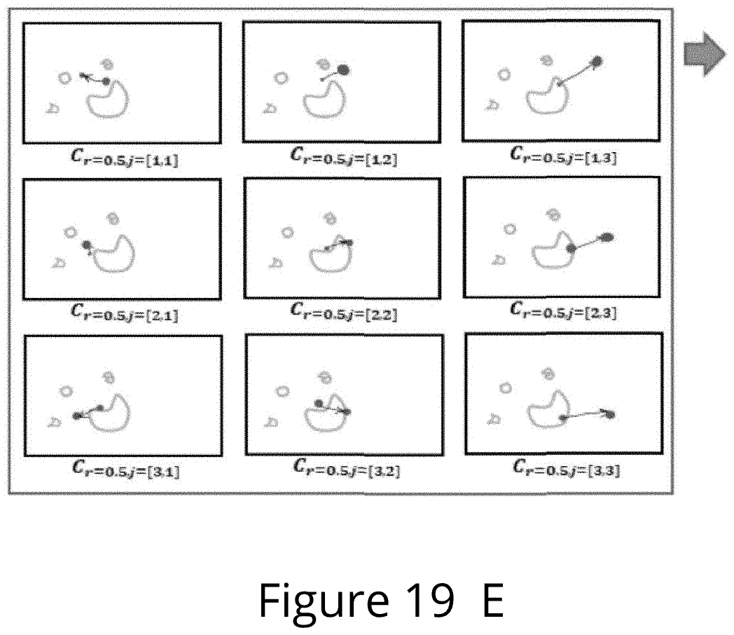

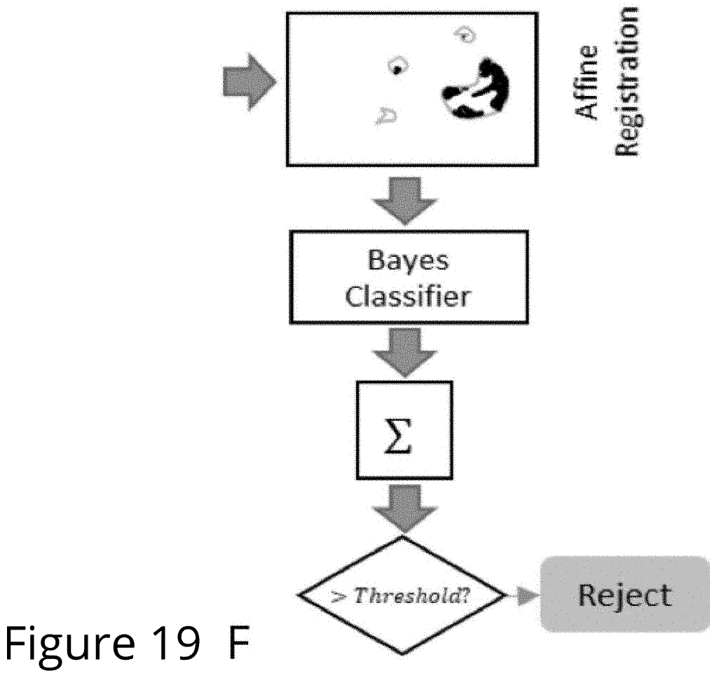

In order to find similarity of registered objects with a probabilistic shape model, a number of points labeled as obj using a Bayes classifier is counted as, Γᵣ=Σ_{X}lᵣ(X), and results in a detection rate at resolution r. If Γᵣ>t res oldᵣ, the registered object is passed to a subsequent iteration at a finer resolution. The block diagram of run-time process as shown was implemented and FIGS. 18 and 19 show two examples one of true-positive detection and one of true-negative detection, respectively.

Though the term iteration is used to describe repeating similar processes at finer resolutions, the overall system described relies upon multiple neural networks, one at each resolution, and as such is implementable in a non-iterative fashion. Advantageously, when implemented in a parallel processing system, each iteration is implementable on separate processors allowing for a pipeline architecture such that each 3D image is processed through all four “iterations” with each image processed in parallel with other images but at different resolutions. Thus, four times as many images are processable though the time from image capture to completion of processing remains similar.

Numerous other embodiments may be envisaged without departing from the scope of the invention.

What is claimed is:

1. A method comprising:
providing a 3D image;
setting current resolution to a first coarse resolution;
setting first current data to a known current data;
iterating an iterative process comprising the steps of:
pre-processing the ultrasound image in accordance with the current resolution;
providing the pre-processed ultrasound image and the current data to a trained system for extracting therefrom objects of interest at the current resolution, the objects of interest extracted with a likelihood above a known threshold;
determining data relating to each extracted object of interest to result in first current data;
when the current resolution is a finest resolution, stopping the iterative process; and
when the current resolution is other than a finest resolution, setting the current resolution to a finer resolution and returning to the start of the iterative process.

2. A method according to claim 1 wherein the 3D image is a medical image of an interior region of a body.

3. A method according to claim 2 wherein the 3D image is one of an Ultrasound image, a CT image, an MRI image, and a PETScan image.

4. A method according to claim 2 wherein the 3D image comprises an ultrasound image and wherein pre-processing comprises denoising the 3D image to produce a 3D image having a current resolution.

5. A method according to claim 2 wherein pre-processing comprises region enhancement of the 3D image to produce a 3D image having a current resolution and with contrast enhancement between regions.

6. A method according to claim 2 wherein objects of interest comprise organs of interest and fluid regions.

7. A method according to claim 6 comprising fluid classification of extracted fluid regions.

8. A method according to claim 2 wherein determining data relating to each extracted object of interest comprises comparing a likelihood that a first extracted object of interest against a known threshold and when the likelihood is below the known threshold excluding data relating to the first extracted object of interest from the first current data.

9. A method according to claim 8 wherein determining data relating to each extracted object of interest comprises comparing a likelihood that a first extracted object of interest is an object of interest against a known threshold and when the likelihood is above the known threshold including data relating to a location and orientation of the first extracted object of interest within the first current data.

10. A method according to claim 1 wherein each iteration of the iterative process relies upon a different trained system, the different trained system trained at a resolution appropriate to a current resolution associated with an iteration during which the different trained system is relied upon.

11. A method according to claim 10, wherein the trained systems comprise neural networks other than deep learning neural networks.

12. A method according to claim 11, wherein the neural networks rely on region specific classifiers.

13. A method according to claim 11, wherein the neural networks rely on some classifiers that vary with resolution.

14. A method according to claim 11, wherein the neural networks rely on some classifiers that remain constant with changes in resolution.

15. A method according to claim 1, comprising: when the iterative process is stopped, identifying potential internal bleeding based on the determined data.

16. A computer aided diagnostic system comprising:
a plurality of trained software processes, each for operating at a higher resolution to extract from three dimensional image data first data relating to an object of interest, the plurality of trained software systems each trained at a different known resolution and each for receiving iteration data based on results of operation of a previous lower resolution trained software system of the plurality of trained software systems, the iteration data providing approximate location information relating to a detected object of interest.

17. A computer aided diagnostic system according to claim 16 wherein each of the plurality of trained software processes is trained for detecting objects of interest and wherein for each detected object of interest of the detected objects of interest, filtering is performed to
determine a likelihood that said detected object of interest is an object of interest and when the likelihood is below a threshold likelihood removing said object of interest from the detected objects of interest.

18. A computer aided diagnostic system according to claim 17 wherein the plurality of trained software processes comprise neural networks.

19. A method comprising:
providing a trainable system comprising a first trainable system and a second other trainable system, each for being provided an initial estimation and for detecting objects of interest within an image;
providing training data comprising an image having a known first resolution and first object of interest data indicative of a presence and a location of an object of interest;
training the first trainable system based on the first object of interest data;
providing a same image having a second resolution finer than the first resolution and second object of interest data indicative of a presence and a location of the object of interest at the second resolution; and
training the second trainable system based on the image and the second object of interest data.

20. A method according to claim 19 wherein trainable system comprising a third trainable system and comprising:
providing a same image having a third resolution finer than the second resolution and third object of interest data indicative of a presence and a location of the object of interest at the third resolution; and
training the third trainable system based on the image and the third object of interest data.
